(19) United States
(12) Patent Application Publication
(10) Pub. No.: US 2023/0215577 A1
(43) Pub. Date: Jul. 6, 2023

(54) BIG DATA PROCESSING FOR FACILITATING COORDINATED TREATMENT OF INDIVIDUAL MULTIPLE SCLEROSIS SUBJECTS

(71) Applicant: Hoffmann-La Roche Inc., Little Falls, NJ (US)

(72) Inventors: Silvia Elena MOLERO LEON, Heredia (CR); Helene Jeanne SAHRI, Basel (CH); Turap TASOGLU, Basel (CH)

(73) Assignee: Hoffmann-La Roche Inc., Little Falls, NJ (US)

(21) Appl. No.: 18/009,217

(22) PCT Filed: Apr. 27, 2021
(86) PCT No.: PCT/US2021/029367

§ 371 Date: Dec. 8, 2022

(30) Foreign Application Priority Data
Jun. 12, 2020 (EP) 20179750.3

(51) Int. Cl. G16H 50/30; G16H 20/00; G16H 10/60

(52) U.S. Cl. CPC G16H 50/30; G16H 10/60; G16H 20/00

(57) ABSTRACT

Disclosed are systems and methods for building and using a data platform to facilitate intelligent selection of treatments for multiple sclerosis and to identify indications for multiple-sclerosis treatments. Various record snapshots of records associated with multiple sclerosis subjects facilitate efficient queries that can be used to explore heterogeneous, unstructured and non-categorical data sets to generate concrete general hypotheses and subject-specific treatment predictions.

### CROSS REFERENCE TO RELATED APPLICATIONS

The present application claims the benefit of the filing date of European Patent Application No. 20179750.3, filed on Jun. 12, 2020, the disclosure of which is hereby incorporated by reference in its entirety for all purposes.

### FIELD

Methods and systems disclosed herein relate generally to systems and methods for using a platform to store and process tagged data pertinent to the determining diagnoses and treatment strategies. Time-sensitive processing may be used to predict temporally relevant data and to generate predicted subject states based on time-series event chains.

### BACKGROUND

Multiple sclerosis (MS) is a common disease with heterogeneous presentation. For example, approximately 1 in 300 individuals in the United States have MS (based on Wallin et al., “The prevalence of MS in the United States” Neurology, 92(1), 2019).

A cause of the disease is not yet known, though various risk factors have been identified. The likelihood of being diagnosed with MS varies depending on (for example) whether a family member was diagnosed with MS (or another autoimmune disease), a latitude of a subject’s residence, whether a subject resides near a coast, whether a subject is female, a subject’s age, a subject’s race, whether a subject has been infected with particular viruses (e.g., Epstein-Barr virus), whether a subject smokes, and whether a subject is obese.

The initial disease presentation, the disease progression and responsiveness to various treatments varies widely across subjects, so much so that some experts question whether MS is actually a set of different diseases. The medical community has identified various disease sub-types and also defined medical tests that may be used to inform treatment decisions. Nonetheless, selected treatments are frequently ineffective.

Clinical trials are frequently conducted to determine an efficacy of a particular new treatment or treatment combination. However, eligibility criteria are frequently restrictive, such that subject groups in trials may not represent the group of patients requesting treatment advice from neurologists. Further, the relatively small size of the subject groups can limit the extent to which data scientists can uncover factors that contribute to efficacy.

Fortunately, the number of treatment options for multiple sclerosis has exploded over the last decade. This is beneficial in that it is less likely that a subject will fail to respond to all treatment options. However, iteratively trying each treatment is now even more impractical with the additional treatment possibilities: It often takes half a year or longer to determine whether the treatment is slowing the rate of progression. During that time, the subject’s multiple sclerosis may be irreversibly progressing more dramatically than it may have if another treatment is used. Thus, it would be advantageous if a system or process were available that could improve subject assessments and treatment selections.

With large amounts of medical data becoming available digitally, systems and methods for personalized medicine are growing. In one example, personalized management and comparison of medical conditions and outcomes based on patient profiles of a community of patients is disclosed by U.S. Pat. Application Publication No. 2015/0324530, and is described as useful with a variety of different medical conditions, including MS. However, the disclosed techniques do not match patients based on prior MS treatments and when these treatments were applied in order to prospectively identify a proposed treatment course that may be effective for a particular MS patient.

Other intelligent medicine approaches have been explored, such as by U.S. Pat. Application Publication No. 2015/0161331, which describes techniques for analyzing, classifying, and matching mass amounts of medical information from many sources and across different regions, with patient medical record data classified into multiple levels of subgroups according to patient clinical parameters, disease templates, treatments, and outcomes, for a wide variety of medical conditions, including MS. When new patients enter the system, the patient’s parameters and disease template are matched against closest subgroups to suggest treatments with potentially favorable outcomes. Again, however, matching of patients based on prior MS treatments and when these treatments were applied in order to prospectively identify a proposed treatment course that may be effective for a particular MS patient is not described.

ALLAM ET AL.: “Patient Similarity Analysis with Longitudinal Health Data”, ARXIV.ORG, 14 May 2020, XP081673418, provides a comprehensive overview of tools and methods used in patient similarity analysis with longitudinal data, discussing the potential for patient similarity analysis for improving clinical decision making. The techniques disclosed, however, do not match patients based on prior MS treatments and when these treatments were applied in order to prospectively identify a proposed treatment course that may be effective for a particular MS patient.

### SUMMARY

In some embodiments, a computer-implemented method is provided. The method includes receiving, at a cloud-based application server, a query that identifies a treatment of multiple sclerosis and querying a data store using an identifier of the treatment, the data store having been populating based at least in part on input received from a distributed set of care-provider entities. The first example’s method further includes receiving, in response to the query, a set of subject identifiers, wherein each subject identifier in the set of subject identifiers indicates that a subject corresponding to the subject identifier received the treatment. The first example’s method further includes – for each subject identifier of the set of subject identifiers – determining, based on data in the data store, a time at which the subject corresponding to the subject identifier initiated the treatment; and extracting, from one or more records associated with the subject identifier: one or more metrics indicative of an outcome of the treatment; and one or more subject attributes The extraction of the one or more metrics was been based at least in part on the time at which the treatment was initiated, and each of the one or more subject attributes reflects a characteristic of a record-corresponding subject or a result of a medical test. The first example’s method still further includes generating a predicted responsiveness of another subject to the treatment based on the extracted metrics and the extracted subject attributes; and outputting a result corresponding to the predicted responsiveness.

In some embodiments, a system is provided that includes one or more data processors and a non-transitory computer readable storage medium containing instructions which, when executed on the one or more data processors, cause the one or more data processors to perform part or all of one or more methods disclosed herein.

In some embodiments, a computer-program product is provided that is tangibly embodied in a non-transitory machine-readable storage medium and that includes instructions configured to cause one or more data processors to perform part or all of one or more methods disclosed herein.

Some embodiments of the present disclosure include a system including one or more processors. In some embodiments, the system includes a non-transitory computer readable storage medium containing instructions which, when executed on the one or more processors, cause the one or more processors to perform part or all of one or more methods and/or part or all of one or more processes disclosed herein. Some embodiments of the present disclosure include a computer-program product tangibly embodied in a non-transitory machine-readable storage medium, including instructions configured to cause one or more processors to perform part or all of one or more methods and/or part or all of one or more processes disclosed herein.

The terms and expressions which have been employed are used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the present invention as claimed has been specifically disclosed by embodiments and optional features, modification and variation of the concepts herein disclosed may be resorted to by those skilled in the art, and that such modifications and variations are considered to be within the scope of this invention as defined by the appended claims.

### BRIEF DESCRIPTION OF THE DRAWINGS

The present disclosure is described in conjunction with the appended figures:

FIG. 1 illustrates a network environment in which the cloud-based application is hosted, according to some aspects of the present disclosure.

FIG. 2 is a flowchart illustrating an example of a process performed by the cloud-based application to distribute condensed subject records to user devices in association with a consult broadcast requesting assistance with treating a subject, according to some aspects of the present disclosure.

FIG. 3 is a flowchart illustrating an example of a process for monitoring the user integration of treatment-plan definitions (e.g., decision trees or treatment workflows) and automatically updating the treatment-plan definitions based on a result of the monitoring, according to some aspects of the present disclosure.

FIG. 4 is a flowchart illustrating an example of a process for recommending treatments for a subject, according to some aspects of the present disclosure.

FIG. 5 is a flowchart illustrating an example of a process for obfuscating query results to comply with data-privacy rules, according to some aspects of the present disclosure.

FIG. 6 is a flowchart illustrating an example of a process for communicating with users using bot scripts, such as a chatbot, according to some aspects of the present disclosure.

FIGS. 7A and 7B depict flowcharts illustrating example processes for building and using snapshot data store representing dynamic and distributed-source data to characterize subpopulations and generate subject-specific predictions.

FIG. 8 depicts a flowchart illustrating an example process for using a snapshot data store to generate high-level treatment-response predictions and/or indications.

FIGS. 9A-9F depict exemplary interfaces to receive inputs to build a multiple-sclerosis data store.

In the appended figures, similar components and/or features can have the same reference label. Further, various components of the same type can be distinguished by following the reference label by a dash and a second label that distinguishes among the similar components. If only the first reference label is used in the specification, the description is applicable to any one of the similar components having the same first reference label irrespective of the second reference label.

### DETAILED DESCRIPTION

### I. Overview

Multiple sclerosis is a heterogeneous disease, with many different types of presentation, manifestations and outcomes. This circumstance presents a challenge for neurologists to select a treatment strategy. However, the unfortunate high prevalence of the disease provides a tool through which big data may be collected and analyzed to support multi-dimensional analyses to inform selection of more effective treatment strategies.

Big data analysis of clinical data is complicated because most clinical-course and patient-attributes correspond to complex non-binary, non-categorical, non-numeric data. Thus, much of this data is represented in unstructured data (e.g., physician notes or radiologist reports), which are difficult to query and aggregate. Some efforts have been made in the medical field to transform complex assessments into categorical or numeric variables, though this can result in information loss and can be inconsistent across actors performing the transformation.

Further, information types that are frequently assessed by neurologists span many data types. Thus, even reviewing a single subject’s files, frequently reviews opening multiple files of differing file types. Some files may include large MRI data, which can include images of various virtual slices of the brain and/or spinal cord, often times repeatedly collected at different perspectives. A neurologist may be comparing information from the collection of files from a recent time period (or snapshot) to corresponding information from a past collection of data corresponding to the subject and then attempting to determine whether any change is acceptable. Notably, almost none of the present treatment options report to completely stop the progression of MS. Rather, they are reported to slow the progression of MS. So the neurologist is tasked with hypothesizing whether any observed change is more pronounced than it would have been if a different treatment had been provided.

Some systems and methods disclosed herein are configured to building and using an MS big data set to facilitate personalized medicine in which treatments are selected for individuals based in part on quantitative multi-variate analysis. In particular, a cloud-based application may provide an interface through which users (e.g., care providers, radiologists, laboratories and/or subjects) can provide input that characterizes a state of a subject. The interface may be configured to (e.g., for some or all types of information) selectively receive input corresponding to a defined structure. Field data presented via the interface may identify the structure. For example, the interface may indicate that disability-related information is to include a score along a particular scale (e.g., the Expanded Disability Status Scale) and/or MRI information is to include a count of a particular type of lesion (e.g., enhanced/non-enhanced as detected in a particular type of scan).

In some instances, the interface may further be configured to receive less structured, semi-structured or unstructured data. The unstructured data may include (for example) physician notes, subject’s textual self-assessment reports, etc. The unstructured data may be processed to identify corresponding structured data (e.g., using natural language processing and/or keyword detection). In some instances, the unstructured data is used as supporting information and kept in unstructured form. For example, in addition to receiving one or more quantitative MRI metrics, the interface may accept MRI images. Though the MRI images may conform with MRI standards, the precise configurations of the data may depend on (for example) the type of machine used and the scans selected.

The cloud-based application may be configured to provide multiple types of interfaces for multiple types of users. For example, a first interface may be availed to physicians and may include one or more pages configured to receive input corresponding to in-person evaluations, in-office assessments, prescription rationale, etc.; one or more second interfaces may be availed to laboratories and/or imaging facilities and may include one or more pages configured to receive medical test results (e.g., blood-test results, MRI images, MRI statistics, etc.); and a third interface may be availed to a subject and may include one or more pages configured to receive self-evaluation responses or self-assessments. For example, a subject interface may be configured to present wellness-related questions and receive corresponding answers. The cloud-based application may further detect, for each user accessing the cloud-based application, a role and permissions. Various permissions may allow a user to input particular types of information (e.g., corresponding to particular fields), to initiate or control particular types of actions (e.g., data processing) and/or to view particular types of data (e.g., corresponding to particular fields and/or particular subjects).

In addition to collecting subject-assessment data that facilitates big-data queries, the cloud-based application may automatically collect or collect via input one or more contextual data points. For example, the cloud-based application may detect a jurisdiction within which a user computer is located and/or a jurisdiction corresponding to a subject’s address (e.g., as input by a user). As another example, the cloud-based application may automatically detect, or detect via input, a time period (e.g., date, time, date period and/or time period) corresponding to an assessment, medical test or input.

The cloud-based application may further receive information about a particular subject. The information may include demographic information, medical-history information, employment information, etc. The information may include an indication as to which medication(s) a subject was taking, during which time period the medication(s) were being taken, any adverse effects experienced during the time period, and potentially a reason why the subject ceased taking the medication. The particular subject information may be detected via input from a user, electronic medical record, automated text recognition, etc.

In some instances, the cloud-based application processes the data to build, for each subject, time series data that associates individual time periods with (for example) any medication being received by the subject, disability level/score, MRI statistics or characteristics, well-being indices, symptom presentation and/or adverse events. In some instances, an event chain may be generated based on the data that includes a series of time stamps and associated clinical, biomarker, medical-test and/or self-assessment metrics. Thus, the event chain may identify which treatments were received by a subject, time periods during which various treatments were received, clinical assessments performed during treatment time periods, results of one or more MRIs received during treatment time periods, subject reports or self-evaluations during treatment time periods, etc.

The cloud-based application may use the subject’s event chain to (for example) generate a prediction corresponding to an effectiveness of a given treatment, predict an effectiveness of a given treatment, predict a prognosis, and/or identify a recommended treatment. For example, the cloud-based application may use the subject’s event chain to identify one or more corresponding filters to impose on the big-data population, to identify one or more sub-population classes and/or to identify one or more nearest neighbors. In some instances, the event chain is input into one or more artificial-intelligence models (e.g., one or more trained machine-learning models) to generate one or more outputs predicting an efficacy and/or outcome of using one or more treatments. For example, each of one or more models may generate an output corresponding to a prediction of switching a subject from a current treatment to another model-corresponding treatment. The output may include (for example) a predicted absolute value or change in a disability score, lesion load, sum of a number of enhancing lesions, etc. A treatment may then be selected based on the output(s).

As another example, the event chain may be used to identify a set of similar subjects who received a given treatment. The similar subjects may be identified by calculating a distance metric between a multi-dimensional event chain of the given subject and each of a set of other subjects who received the given treatment and identifying a subset of the set of other subjects who are associated with distance metrics below a particular threshold. A prediction of the given subject’s responsiveness to the given treatment may be defined based on the responsiveness of the subset of subjects to the given treatment (e.g., an average or median disability progression within a time period, average or median number of relapses within a time period, average or median number of new lesions within a time period, etc.).

An interface may be configured to receive input that identifies, for each treatment selection made in association with a given subject, an identifier of the treatment (e.g., name), a date or time period at which the treatment was initiated, any adverse events experienced by the subject in association with the treatment (e.g., and when such adverse events were experiences), and/or a date or time period at which the treatment was terminated. The subject’s event chain may be updated to reflect some or all of the treatment information.

In some instances, data associated with a given subject (e.g., a subject’s event chain) may be used to identify one or more corresponding subjects. The corresponding subject(s) and the given subject may be similar and/or associated with one or more same or similar attributes. For example, each of the corresponding subjects may have received a same particular treatment as one that the subject is receiving, will receive or is considering. The corresponding subjects and the given subject may be associated with a similar or same type of multiple sclerosis, similar or same disability scores, similar or same treatment histories, etc. Which attributes are to influence definition of the corresponding subject set may be defined via user input, predefined or learned by a model.

As one illustration, a user of the cloud-based application may initiate a query by selecting field constraints and identifying a subject (with relapse-remitting multiple sclerosis who is fully ambulatory may have recently transitioned from an interferon beta treatment to a ocrelizumab treatment). A data store may be queried to identify other subjects for which the field constraints are satisfied in that they were also fully ambulatory and diagnosed with relapse-remitting multiple sclerosis at a time when they also transitioned from an interferon beta treatment to a ocrelizumab treatment. Though in this exemplary instance, the field constraints correspond to actual attributes of the subject, in some situations, one or more constraints may correspond to a potential attribute (e.g., a treatment being considered for the subject). Notably, this query may be backwards looking so as to identify subjects who were previously associated with various characteristics that are presently associated with the given subject.

To facilitate performing retrospective queries, the cloud-based application may generate and store snapshots for each treatment-transition point (at which a treatment is initiated, changed or terminated) for each subject. The snapshot can include last-reported information and/or calculated information. A snapshot may include fields and/or information described herein, such as a most recent treatment, one or more MRI statistics, one or more ambulatory metrics (e.g., whether the subject uses a cane, whether the subject uses bilateral walking supports, whether the subject uses a walker, and/or whether the subject uses a wheelchair), a type of multiple sclerosis, etc. For example, if a subject changed medications in July 2017, information about many other fields may not have been provided at this same time. However, previously received information can be queried to detect (for example) that a most recent treatment prescribed was in January 2016 and was fingolimod, that a most recent MRI administered in April 2017 corresponded to a set of relevant MRI statistics, data entry corresponding to a March 2017 office visit indicated that the subject was using a cane, and a most-recent diagnosis from September 2008 was that ore relapse-remitting multiple sclerosis. Thus, this information is essentially pulled through to the July 2017 snapshot.

Thus, the snapshots may facilitate performing retrospective multi-dimensional queries using constraints that need not be concurrently observed. For each corresponding subject, a date on which the treatment was initiated can be set as time zero. One or more data points associated with the corresponding subject and subsequent time points (e.g., up until and including a date on which the corresponding subject terminated use of the particular treatment) can be collected. The subsequent data points may indicate (for example) a mobility or ambulatory level, disability level, one or more MRI metrics, adverse-event indicators and/or any treatment-termination indicator. Aggregate statistics can then be generated based on the subsequent data points and potentially based on snapshot data.

For example, aggregate statistics can include a distribution of time periods for which the corresponding subjects remained on the particular treatment. As another example, aggregate statistics can include, for each time period, a statistic (e.g., median, mean or mode) characterizing a number of new lesions detected across corresponding subjects; characterizing a number of new enhancing lesions; characterizing a change in total volume of a particular type of lesion; characterizing a change in disability level; characterizing a change in ambulatory level; characterizing a change in a well-being index; characterizing a presence, prevalence or severity of adverse events), and/or characterizing a probability or number of relapses. Which post-treatment information is tracked may be determined based on input corresponding to the query or based on predetermined configurations.

In some instances, the corresponding subjects can be split into two or more groups, and aggregate statistics can be generated for each group. For example, a first group may include the corresponding subjects who continued receiving the treatment for at least a predetermined amount of time, such as for 18 months), and a second group may include the corresponding subjects who switched off of the treatment prior to the predetermined amount of time. As another example, a first group may include the corresponding subjects who received the treatment for at least a first period of time (e.g., for at least 6 months) and whose Expanded Disability Status Scale (EDSS) score stayed the same or changed by less than 1.5 points over the period of a second period of time (e.g., within 12 months), while a second group may include the corresponding subjects who received the treatment for at least the first period of time and whose EDSS score changed by 1.5 or more points over the second period of time.

With regard to either example, for each of the groups, aggregate statistics that are generated may represent information at baseline (time zero) for subjects in the group, such as demographic information (e.g., age distribution, sex distribution), medical-history information (e.g., prevalence of comorbidity), baseline MRI statistic (e.g., lesion load, number of total lesions, number of enhancing lesions), and/or disability metric (e.g., EDSS score or ambulatory level). Thus, the aggregate statistics can be used to predict what types of subject attributes and/or contexts are predictive of treatment efficacy and/or of a positive prognosis while on the treatment. A care provider or subject may use the prediction and particular attributes of the subject to determine whether to select the treatment or continue use of the treatment. In some instances, a prediction of efficacy and/or prognosis may be generated for the subject for each of multiple treatment strategies (e.g., associated with different corresponding subject populations), and a treatment can be selected for the subject based on the predictions.

In some instances, after the given subject has initiated the treatment, one or more data points may be tracked for the given subject and compared to aggregate statistics for the corresponding subjects or one or more groups within the corresponding subjects. For example, fourth months after treatment initiation, a change in T2 lesion load, a change in EDSS score and a number of enhancing lesions can be determined for the given subject. A model (e.g., classifier) or multi-variate analysis may be used to predict whether the subject-specific data points align more strong with a “responder” population sub-group or with a “non-responder” population sub-group (e.g., where the responder sub-group was associated with longer use of the treatment, smaller MRI-based progression, smaller disability progression). A care provider or subject may use the prediction to determine whether to change a treatment strategy for the subject or consider the same.

In some instances, a query may be performed that includes constraints corresponding to a symptom set, disability score and/or set of test results associated with a given subject. The symptom set, disability score and/or test results may (but need not) be insufficient to support a multiple-sclerosis diagnosis. A population of other subjects having had similar symptoms, disability scores and/or test results may be identified. The query may be performed to as to further impose negative constraints (e.g., indicating that one or more other symptoms are not experienced). The population may be sub-divided based on treatment approaches that were used. For each group within the population, statistics may be generated that indicate (for example) a likelihood of transitioning to another sub-type of multiple sclerosis or to a diagnosis of multiple sclerosis (e.g., as a function of time or within a period of time); a change in disability scores (e.g., an median change as a function or time or a probability of a worse score occurring within a period of time); a probability of changing treatment as a function of time, etc. A care provider may then recommend a treatment that is associated with a relative low likelihood of transitioning to another sub-type, a relatively small change (or no change) in disability score, and/or a relatively small probability of changing treatments.

The cloud-based application may operate to implement data-privacy protocols that enable an entity to transmit and/or receive one or more data records or other information characterizing subjects (e.g., experiencing medical symptoms and/or having a possible or confirmed diagnosis of a medical condition) with external entities, while satisfying the constraints imposed by data-privacy rules across various jurisdictions. The cloud-based application can be configured to algorithmically assess data-privacy violations and automatically omit, obfuscate or otherwise modify data records to comply with data-privacy rules. For example, the cloud-based application may be configured to determine whether aggregate statistics generated based on data associated with a set of corresponding subjects may risk revealing identifying information. Such information may be more likely to be revealed when (for example) constraints for identifying the corresponding subjects are particularly narrow, a size of the population of corresponding subjects (or sub-group there) is particularly small, a number of aggregate statistics availed or presented is particularly high, etc. To illustrate, if a query includes a zip-code constraint along with three other constraints and a resulting population of subjects includes only 3 subjects, even presentation of statistics generated based on data from all 3 subjects may risk revealing personally identifiable information.

If it is determined that responding to a query risks revealing personally identifiable information, the cloud-based application may determine whether a user is authorized to view the information for subjects that would be represented in the data. If not, the cloud-based application may reject the query or potentially modify the query to include less restrictive constraints.

Some embodiments of the present disclosure provide a technical advantage over conventional systems by providing a cloud-based application configured to exchange subject information with external entities without violating data-privacy rules. The cloud-based application facilitates selective collection and processing of data to arrive at a data set that is relatively consistent in content and format across subjects and other users. The data representations can further facilitate data queries, in that fields for which constraints can be identified can be identifiable. Queries for subject populations may then be performed capitalizing on logic operands and/or basic searches rather than performing natural-language-processing queries. Further, transforming subject records into event streams and/or generating snapshots at particular significant events can facilitate performing queries when various data points are not temporally aligned. The use of machine-learning models (e.g., classifiers) and the population data can facilitate improving the predicting factors influencing treatment efficacy and/or tolerability.

While the disclosure above describes a cloud-based application configured to perform intelligent functionality with respect to facilitating diagnosis and treatment of multiple sclerosis, the cloud-based application may be configured to identify potential diagnoses or potential treatments for any disease, condition, area of study, or disorder, including, but not limited to, oncology, including cancers of the lung, breast, colorectal, prostate, stomach, liver, cervix uteri (cervical), esophagus, bladder, kidney, pancreas, endometrium, oral, thyroid, brain, ovary, skin, and gall bladder; solid tumors, such as sarcomas and carcinomas, cancers of the immune system including lymphomas (such as Hodgkin or non-Hodgkin), and cancers of the blood (hematological cancers) and bone marrow, such as leukemias (such as Acute lymphocytic leukemia (ALL) and Acute myeloid leukemia (AML)), lymphomas, and myeloma. Additional disorders include blood disorders such as anemia, bleeding disorders such as hemophilia, blood clots, ophthalmology disorders, including diabetic retinopathy, glaucoma, and macular degeneration, neurological disorders, including Parkinson’s, disease, spinal muscular atrophy, Huntington’s Disease, amyotrophic lateral sclerosis (ALS), and Alzheimer’s Disease, autoimmune disorders, including multiple sclerosis, diabetes, systemic lupus erythematosus, myasthenia gravis, inflammatory bowel disease (IBD), psoriasis, Guillain-Barre syndrome, Chronic inflammatory demyelinating polyneuropathy (CIDP), Graves’ disease, Hashimoto’s thyroiditis, eczema, vasculitis, allergies and asthma.

Other diseases and disorders include but are not limited to kidney disease, liver disease, heart disease, strokes, gastrointestinal disorders such as celiac disease, Crohn’s disease, diverticular disease, Irritable Bowel Syndrome (IBS), Gastroesophageal Reflux Disease (GERD) and peptic ulcer, arthritis, sexually transmitted diseases, high blood pressure, bacterial and viral infections, parasitic infections, connective tissue diseases, celiac disease, osteoporosis, diabetes, lupus, diseases of the central and peripheral nervous systems, such as Attention deficit/hyperactivity disorder (ADHD), catalepsy, encephalitis, epilepsy and seizures, peripheral neuropathy, meningitis, migraine, myelopathy, autism, bipolar disorder, and depression.

### II. Summary of MS Sub-Types, Diagnosis Protocol, Pertinent Medical Tests, Progression Assessment and Available Treatments

### II.A. Types of Multiple Sclerosis

The medical community has defined multiple types of MS, which can influence treatment selection and inform prognoses.

### II.A.1. Relapse-Remitting Multiple Sclerosis (RRMS)

In 85-90% of cases, multiple sclerosis first presents as a relapse-remitting multiple sclerosis (RRMS) type. RRMS patients experience discrete exacerbations (or “relapses”) during which new neurological symptoms appear or during which old symptoms worsen. The prominent mechanism hypothesis is that exacerbations are a result of an autoimmune cascade during which autoreactive leukocytes traverse the blood-brain barrier from the periphery and attack the myelin protective layers on neuron projections in the central nervous system. The autoimmune cascade is thought to predominantly involve T cells but to also involve some B cells (e.g., to recruit macrophages, activate the complement pathway and/or produce costimulatory molecules that influence T cell differentiation). Another theory is that the attack is performed by myelin-specific T cells with malfunctioning immunoregulatory mechanisms.

Each exacerbation may result in one or more physiological symptoms. A physiological symptom may be a symptom associated with one of eight functional systems. Functional systems and select associated symptoms include the pyramidal system (symptoms: muscle weakness or difficulty moving limbs); cerebellar system (balance problems or tremor); brainstem system (difficulties with speech or swallowing); sensory system (numbness or tingling); bowel or bladder system (incontinence); visual system (blurriness or blindness); cerebral system (deficits in memory, multi-tasking or thinking); and other. Between exacerbations, symptoms may partly or completely disappear. Full recovery from relapses is more likely when the relapse occurred closer to a diagnosis date (as compared to later time periods). Early in the disease, relapses typically involve the pyramidal, cerebellar, sensory, or visual functional systems. Later in the disease, relapses often involve the brainstem, bowel or bladder, or cerebral functional systems.

### II.A.2. Active or Inactive Secondary Progressive Multiple Sclerosis (SPMS)

If left untreated, approximately 90% of patients with RRMS will transition to secondary progressive multiple sclerosis (SPMS) within 25 years (with about 50% transmitting within 10 years). There is no general agreement in the medical community as to what precise indicators mark a transition from RRMS to SPMS.

SPMS patients who also continue to experience symptomatic relapses and inflammation are deemed to have active SPMS, while those without relapses are characterized as having inactive SPMS. However, regardless, these patients typically experience gradual and generally monotonic decline in function as a result of nerve damage or loss. Stated differently, the decline observed in SPMS patients is generally understood to be primarily due to neurodegeneration, not due to discrete immunological attacks.

### II.A.3. Primary Progressive Multiple Sclerosis (PPMS)

Some MS patients are never diagnosed with the RRMS form and instead are initially diagnosed with Primary Progressive Multiple Sclerosis (PPMS). As with SPMS, these patients generally experience gradual and sustained worsening of symptoms and brain injury. With regard to both SPMS and PPMS, the gradual function loss may occur over a time period of months to years, making it difficult to detect. Most people with PPMS initially present with motor symptoms (while sensory and/or optical symptoms are more prevalent for RRMS). The prominent criteria for a PPMS diagnosis is that functional-system impairment occurs gradually and independently of discrete relapses (such that the gradual worsening is not a result of any residual impairment triggered by relapses).

### II.A.4. Progressive-Relapsing Multiple Sclerosis

Some people experience symptomatic relapses and/or enhancing lesions after a diagnosis of MS. In these cases, the patient is diagnosed with Progressive-Relapsing MS, as the initial diagnosis of progressive MS is not discarded..

### II.A.5. Clinically Isolated Syndrome (CIS)

Even though, clinically Isolated Syndrome (CIS) is considered a sub-type of multiple sclerosis, CIS is actually distinct from multiple sclerosis. A diagnosis of CIS indicates that a subject experienced a first neurological symptom for at least 24 hours that is isolated in time and not due to another medical condition (e.g., stroke or Lyme’s disease). If the subject recalls one or more other prior neurological symptoms or if an MRI identifies old (non-enhancing) lesions, the subject is to be diagnosed with multiple sclerosis, not CIS.

Subjects with CIS may, or may not, subsequently experience additional symptoms and receive a multiple-sclerosis diagnosis. CIS patients may receive select multiple-sclerosis medications, which may reduce the probability that or extend a time at which the subject is diagnosed with multiple sclerosis.

### II.B. Diagnosis of Multiple Sclerosis

Patient reports of symptoms to care providers, and functional assessments performed by care providers is highly relevant when determining whether to diagnose a patient with multiple sclerosis and/or when diagnosing a patient with a particular sub-type of multiple sclerosis. However, results of several other medical tests may be highly influential in the diagnosis.

### II.B.1. Medical Tests Potentially Informative for Multiple Sclerosis Diagnosis

### II.B.1.A. Magnetic Resonance Imaging

Magnetic Resonance Imaging (MRI) machines include large magnets that generate strong magnetic fields. These fields cause protons in the body to align with the field. A resonance-frequency radiofrequency (RF) field causes the protons to spin out of equilibrium against the magnetic field. When the RF field turns off, the protons release energy while realigning with the magnetic field. An MRI machine includes a receiving coil to measure this energy release. Different types of biological structures will result in different energy-release profiles (e.g., identifying a time elapsed to return to an equilibrium state). Two operation settings include a repetition time and an echo time. The repetition time is the time between successive RF pulses. Long repetition times enable all protons to realign with the magnetic field before a next pulse, whereas short repetition times result in many protons only partly re-aligning. The echo time indicates when signals produced by the protons are measured. Longer echo times make it more likely that protons in gray and white matter will go out of phase, which can result in longer signals. Meanwhile, fluids are less sensitive in this respect, so their signals will remain stronger.

MRI images can include T1-weighted images (T1 images), T2-weighted images (T2 images) or FLAIR images. T1 images are produced in response to short echo times (e.g., 14 ms) and short repetition times (e.g., 500 ms). In T1 images, white matter (e.g., axons) is light, gray matter (e.g., nerve cell bodies and dendrites) is gray, the spinal cord is gray, cerebrospinal fluid (CSF) is dark, and inflammation is dark. Black holes will appear in T1 images as hypointense (dark) areas. When a contrast agent (e.g., gadolinium) is administered to a subject, it may pass through the blood-brain barrier only if this barrier had been recently disrupted and can leak into the recently formed lesions. A T1 image will then depict these lesions as bright areas.

T2 images are produced in response to long echo times (e.g., 90 ms) and long repetition times (e.g., 4000 ms). In T2 images, white matter is dark gray, gray matter is light gray, the spinal cord is light gray, CSF is bright, and inflammation is bright. T2 images may thus be used to detect new and old lesions (which will appear bright). FLAIR images are produced in response to even longer echo times (e.g., 114 ms) and even longer repetition times (e.g., 9000 ms). In FLAIR images, white matter is dark gray, gray matter is light gray, the spinal cord is light gray, CSF is dark, and inflammation is bright. FLAIR images allow better visibility of lesions adjacent to the CSF as compared to T2 images.

MRI images of the brain and/or spinal cord can be used to facilitate a diagnosis of multiple sclerosis, to facilitate a diagnosis of a particular type of multiple sclerosis, to facilitate characterizing a progression of multiple sclerosis in a subject, to facilitate characterizing a degree to which a treatment is effectively treating a subject, and/or to facilitate determining whether to change a treatment strategy for a subject. The number, location, size and shape of the lesions may inform one or more of these decisions. For example, “Dawson fingers” are common lesions occurring in patients with RRMS. Dawson fingers are multiple lesions that are ovoid shape, located near the ventricle-based veins in the brain (deep medullary veins). While lesions may present in any CNS white matter, multiple sclerosis lesions frequently occur in the periventricular white matter, cerebellum, brainstem and spinal cord.

MRI analysis frequently involves determining whether MRI images depict any contrast-enhancing lesions, as such lesions typically indicate that the subject’s multiple sclerosis was recently active. T2-weighted lesions are also frequently used to measure total lesion volume. T2 disease-burden metrics measured early in the disease process (e.g., in CIS or RRMS) can provide information about a longer term disability of a subject and/or disease progression.

Further, MRIs may be used to identify a cross-sectional area (e.g., at a segment of the spinal cord) or volume (e.g., of the brain), which can be used to estimate an extent of atrophy in a subject. The atrophy may be estimated by comparing area/volume metrics generated based on a recent MRI of the subject to those generated based on an older MRI of the subject. The atrophy may further be estimated by comparing area/volume metrics generated based on a recent MRI of the subject to statistics generated based on a comparable population. Atrophy statistics may be used to inform a determination relating to MS diagnosis and/or treatment decision.

As further described below, the presence of lesions in multiple anatomical areas and/or time-separated appearance of lesions is indicative of multiple sclerosis disease. Time-separated appearance of lesions may be detected by comparing MRIs collected at different time points or by detecting that at least one lesion is contrast enhanced (indicating recent inflammation) and that at least one lesion is not contrast enhanced.

### II.B.1.B. Cerebrospinal Fluid Analysis

The cerebrospinal fluid (CSF) of most subjects with multiple sclerosis (and of many subjects with other inflammatory medical conditions) include relatively high concentrations of Immunoglobulin G (IgG) as compared to the general population. IgGs appear to be a secondary effect of multiple sclerosis but still provide a useful biomarker.

Thus, analysis of CSF is frequently performed during a diagnostic stage of multiple sclerosis. CSF is collected from a subject via a spinal tap. One or more laboratory tests (e.g., protein electrophoresis, Western blot, or a combination of isoelectric focusing and silver staining) are performed to detect proteins in each of a CSF sample and potentially in a serum sample (e.g., a diluted serum sample).

Detecting IgG bands in the CSF is consistent with a multiple sclerosis diagnosis. Additionally or alternatively, detecting oligoclonal bands in the CSF and serum along with additional bands in the CSF is also consistent with a multiple sclerosis diagnosis. Any of these detections are further predictive of whether a subject with CIS will convert to clinically definite MS.

An IgG CSF Index is defined as the ratio of IgG relative to albumin in the CSF as compared to the ratio of IgG relative to albumin in serum. Values higher than 1 indicate that the central nervous system is producing IgG. The IgG CSF Index can also be used to generate an IgG synthesis rate.

### II.B.1.C. Visually Evoked Potentials

Myelination of axons increases the speed at which action potentials move along the axons. Thus, if the myelin coasting is attacked via an inflammatory multiple sclerosis attack, the conductance of nerve impulses may subsequently be slowed. One test that can be informative in the diagnosis of multiple sclerosis is evoking action potentials and measuring the speed of their transmission. Typically, for this assessment, potentials are evoked by presenting a particular visual stimulus (e.g., a binary, dynamic checkerboard) while brain signals (e.g., in the occipital cortex) are non-invasively recorded (e.g., via electroencephalography). However, auditory or somatosensory stimuli may alternatively be used. The evoked potential includes two negative peaks and one positive peak (between the two negative peaks). The magnitude and/or time of each of these peaks can be informative of diagnosis and/or prognosis of multiple sclerosis. For example, a symptom of multiple sclerosis called optic neuritis can result in loss of the positive peak and/or highly attenuated responses.

### II.B.2. Criteria Used for Multiple Sclerosis Diagnosis

There is no single test or single assessment that reliably determines whether a subject has multiple sclerosis. In some instances, a single test result (e.g., set of MRI images) may be sufficient for a multiple-sclerosis diagnosis, but that test result is not reliably deterministic across subjects and circumstances.

Many medical providers characterize the diagnosis of multiple sclerosis as a diagnosis of exclusion, in that the clinician is tasked with prescribing tests and performing analyses that would rule out potential other causes for observed abnormalities. For example, many neurological symptoms and/or MRI results may be explainable by infectious disease (e.g., Lyme’s disease), spinal cord compression, vitamin B₁₂ deficiency or non-MS inflammatory de-myelinating disease (e.g., sarcoidosis, systemic lupus erythematosus, neuromyelitis optica or acute disseminated encephalomyelitis). If no such alternative explanations can be identified, a diagnosis of multiple sclerosis is appropriate.

A diagnosis of exclusion, unfortunately, is not a quick diagnosis. While subjects wait to be able to receive various tests, wait for the test results, and wait for their care providers’ assessments, it is possible that their multiple sclerosis just continues to progress. Various protocols and disease characterizations have attempted to take such situations into consideration. For example, most criteria for diagnosis of multiple sclerosis does not depend on ruling out all potential alternative causes (although some people may then discover – years after their multiple-sclerosis diagnosis – that they, in fact, have a different medical condition); further, the establishment of Clinically Isolated Syndrome (CIS) has allowed physicians to prescribe some multiple sclerosis medications that may slow progression, despite the fact that a confirmed multiple-sclerosis diagnosis has not yet been reached.

### II.B.2.A. McDonald Criteria

A prominent criteria for diagnosing multiple sclerosis is the McDonald Criteria. An underpinning of this criteria is that the physician is to generally have evidence of separation in space and time to diagnosis a subject with multiple sclerosis. Separation in space may correspond to symptoms affecting different parts of the body or different functional systems or lesions present in different parts of the central nervous system. Separation in time may correspond to symptoms and/or lesions appearing at different times, which may be detectable via multiple assessments (e.g., multiple MRIs or multiple clinical/symptomatic assessments) performed at different times or by detecting some contrast-enhancing lesions (e.g., indicating recent inflammation) and some non-enhanced lesions (e.g., indicating that they were not recently formed).

The McDonald criteria has emerged as a standard frequently used for diagnosis with multiple sclerosis. Under this criteria, any of the following circumstances results in a multiple sclerosis diagnosis, and a multiple sclerosis diagnosis is not to be otherwise made.
Two or more relapses and symptomatic clinical evidence of two or more lesions
Two or more relapses, clinical evidence of a lesion in the central nervous system, and one or more lesions typical of multiple sclerosis;
Two or more relapses, clinical evidence of a lesion in the central nervous system, and another relapse indicating injury to another part of the central nervous system;
One relapse, clinical evidence of two or more lesions (e.g., impairment in two or more functional systems or two or more parts of the body); and disparate immune-system activity as evidenced by oligoclonal bands as identified based on multiple time-separated spinal taps;
One relapse, clinical evidence of two or more lesions (e.g., impairment in two or more functional systems or two or more parts of the body); and MRI evidence of a new lesion since a previous scan;
One relapse, clinical evidence of two or more lesions (e.g., impairment in two or more functional systems or two or more parts of the body); and a further symptomatic relapse;
One relapse, clinical evidence of one lesion, one or more MRI lesions typical of multiple sclerosis;
One relapse, clinical evidence of one lesion, another relapse showing activity in a different part of the central nervous system as indicated by oligoclonal bands, an MRI depicting a new lesion since a previous scan or a new relapse;
One relapse, clinical evidence of one lesion, one or more MRI lesions typical of multiple sclerosis; or
Gradual progression of neurological symptoms typical of multiple sclerosis for one year plus any two of: at least one brain lesion typical of multiple sclerosis, two or more lesions in the spinal cord, or oligoclonal bands in the spinal fluid.

### II.C. Assessments of Multiple Sclerosis Progression

After a subject is diagnosed with multiple sclerosis, periodic appointments with a neurologist are typically made to assess various functions and convey current or recent symptoms. In some instances, these appointments are scheduled at regular intervals (e.g., every 6 months). In some instances, a subject requests one or more appointments to discuss new symptomatic exacerbations, medication side effects, etc. Neurologists frequently also recommend that subjects periodically receive MRIs. Again, the MRIs may be periodically scheduled to generally assess progression, or they may be taken to determine whether a new potential or actual symptom likely corresponds to an exacerbation or to determine whether a current treatment has been effective in slowing or halting progression of the disease.

Thus, physicians may determine whether and/or an extent to which a subject’s multiple sclerosis is progressing based on one or more of: a frequency of new symptoms; a severity of new symptoms; a worsening of old symptoms; a change in a number of functional systems for which symptoms are experienced; a frequency of new lesion development; a change in lesion load; a change in atrophy; and/or subject self-assessments. Various scales have been developed to more objectively assess a progression of multiple sclerosis. Some scales are designed to assess well-being of a subject and are based on subjects’ responses to a series of questions.

Some scales are based on physicians’ assessments of a subject’s functional abilities. One such functionality-based scale is the Expanded Disability Status Scale. This scale is largely dependent on a subject’s ambulatory ability. The scale ranges from 0 to 10 and is discretized by 0.5 increments. Scores between 0 and 4.5 are distinguished based on a quantity of functional systems for which a subject is experiencing a symptom or disability and a severity of the symptom/disability. Scores between 4.5 and 7.5 are distinguished based on how far a subject can move and whether and/or whether and/or which types of walking aids are needed for the movement. Scores between 7.5 and 9.5 are distinguished based on a degree to which a subject is restricted to bed and retains any function. A score of 10 is indicative of death due to multiple sclerosis.

### II.D. Multiple Sclerosis Treatments

Many new multiple sclerosis treatments have been brought to the market in recent years. The treatments can be differentiated based on their mechanisms (many of which are only partly understood), their routes of administration and whether the treatment is characterized as a first-line approach or a second- or third-line approach.

Injectable medications include interferon-beta and glatiramer acetate. Interferon-beta is an immunomodulator that reduces CD4+ and CD8+ T-cell reactivity. Interferon-beta is marked under three trade names (Avonex®, Betaseron® and Rebif®), which differ with regard to dose formulation, route of administration and frequency of use. Glatiramer acetate is an immunomodulator that resembles myelin, binds to the maj or histocompatibility complex and reduces binding of T cells with antigens.

Recently, many more oral medications have become available, include teriflunomide, monomethyl fumerate, fingolimod, siponimod, cladribine, dimethyl fumerate, diroximel fumarate, laquinimod and ozanimod. Teriflunomide inhibits mitochondrial enzyme dihydroorotate dehydrogenase. Dimethyl fumerate, monomethyl fumerate and diroximel fumarate act through a mechanism still currently being explored to induce lymphocytopenia. Fingolimod and siponimod modulate the sphingosine 1-phosphate (S1P), with siponimod and ozanimod are selective for S1P₁ and S1P₅, while fingolimod is a nonselective S1P modulator. Cladribine depletes CD4⁺ and CD8⁺ lymphocytes. Laquinimod interferes with T cell migration to the central nervous system and decreases IL-17 levels.

Still other medications are delivered intravenously, including ocrelizumab, ofatumamab, ublituximab, rituximab, natalizumab, alemtuzumab, and mitoxantrone. Ocrelizumab, ofatumumab, ublituximab and rituximab target CD20⁺ B cells via complement-dependent cytotoxic effects. Natalizumab is a monoclonal antibody that targets lymphocytes expressing CD52. Alemtuzumab is a monoclonal antibody that binds to CD52 on T and B cells to prime the T and B cells for destruction. Mitoxantrone is a type II topoisomerase inhibitor that suppresses immune-cell proliferation, impairs antigen presentation, enhances T-cell suppressor function and inhibits B cells.

Yet still another treatment approach is autologous hematopoietic stem cell therapy. This approach involves removing some of a patient’s bone marrow stem cells (released into the blood in response to a chemotherapy agent), administering a multi-day chemotherapy treatment (to deplete most or all of the subject’s immune cells) and to administer the stored stem cells (to facilitate recovery).

Medications can also be distinguished based on for which sub-type(s) of multiple sclerosis the medication is approved to treat. Most approved multiple sclerosis medications are approved to treat RRMS. Medications approved to treat SPMS include mitoxantrone, cladribine and siponimod. The only medication approved in the US and Europe as of June 2020 for treating PPMS is ocrelizumab.

Even more treatments are available to treat symptoms of multiple sclerosis (rather than the disease itself). For example, dalfampridine can be used to improve walking, tolterodine can be used to treat incontinence, or baclofen can be used to treat spasticity. Further, solumedrol (intravenous), prednisone (oral) and/or methylprenisolone (oral) are frequently used to treat multiple sclerosis relapses (e.g., to speed recovery from the relapse and/or increase the probability of a full recovery from the relapse).

Each treatment can be used as a first line, second line or third line treatment. In general, a second-line treatment is one to be administered after determining that one or more first-line treatments have failed, ceased being effective for a subject or causes intolerable adverse events for a subject. Similarly, a third-line treatment is one to be administered after determining that one or more second-line treatments have failed, ceased being effective for a subject or causes intolerable adverse events for a subject. Different physicians may make different decisions as to which treatments are characterized as first-line treatments. These decisions may be based on adverse-event profiles, as higher risks of adverse effects and/or risks of more severe adverse effects may be more acceptable if other treatment options have failed. According to the MS Foundation, treatments typically used as first-line treatments include teriflunomide, interferon beta, glatiramer acetate, ocrelizumab, peginterferong beta and dimethyl fumerate. Treatments typically used as second- or third-line treatments include fingolimod, alemtuzumab, cladribine, siponimod, mitoxantrone and natalizumab.

### III. Network Environment for Hosting the Cloud-Based Application Configured with Intelligent Functionality

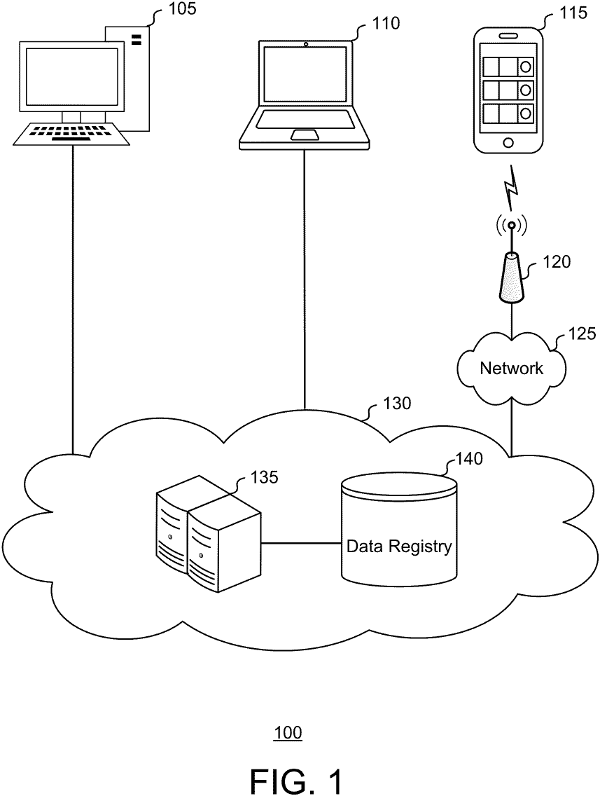

FIG. 1 illustrates network environment 100, in which an embodiment of the cloud-based application is hosted. Network environment 100 may include cloud network 130, which includes cloud server 135 and data registry 140. Cloud server 135 may execute the source code underlying the cloud-based application. Data registry 140 may store the data records ingested from or identified using one or more user devices, such as computer 105, laptop 110, and mobile device 115.

The data records stored in data registry 140 may be structured according to a skeleton structure of fixed parts (e.g., data fields). Computer 105, laptop 110, and mobile device 115 may each be operated by various users. For example, computer 105 may be operated by a physician, laptop 110 may be operated by an administrator of an entity, and mobile device 115 may be operated by a subject. Mobile device 115 may connect to cloud network 130 using gateway 120 and network 125. In some examples, each of computer 105, laptop 110, and mobile device 115 are associated with the same entity (e.g., the same hospital). In other examples, computer 105, laptop 110, and mobile device are associated with different entities (e.g., different hospitals). The user devices of computer 105, laptop 110, and mobile device 115 are examples for the purpose of illustration, and thus, the present disclosure is not limited thereto. Network environment 100 may include any number or configuration of user devices of any device type.

In some embodiments, cloud server 135 may obtain data (e.g., subject records) for storing in data registry 140 by interacting with any of computer 105, laptop 110, or mobile device 115. For example, computer 105 interacts with cloud server 135 by using an interface to select subject records or other data records stored locally (e.g., stored in a network local to computer 105) for ingesting into data registry 140. As another example, computer 105 interacts with an interface to provide cloud server 135 with an address (e.g., a network location) of a database storing subject records or other data records. Cloud server 135 then retrieves the data records from the database and ingests the data records into data registry 140.

In some embodiments, computer 105, laptop 110, and mobile device 115 are associated with different entities (e.g., medical centers). The data records that cloud server 135 obtains from computer 105, laptop 110, and mobile device 115 may be stored in different data registries. While the data records from each of computer 105, laptop 110, and mobile device 115 may be stored within cloud network 130, the data records may not be intermingled. For example, computer 105 cannot access the data records obtained from laptop 110 due to the constraints imposed by data-privacy rules. However, cloud server 135 may be configured to automatically obfuscate, obscure, or mask portions of the data records when those data records are queried by a different entity. Thus, the data records ingested from an entity may be exposed to a different entity in an obfuscated, obscured, or masked form to comply with data-privacy rules. As an illustrative example, a physician or other medical professional can input a subject’s symptoms, which potentially relate to multiple sclerosis. The symptoms may be stored within cloud network 130 as part of the subject record associated with the subject.

Once the data records are collected from computer 105, laptop 110, and mobile device 115, the data records may be used as training data to train machine-learning or artificial-intelligence models to provide the intelligent analytical functionality described herein. The data records may also be available for querying by any entity, given that when a user device associated with an entity queries data registry 140 and the query results include data records originating from a different entity, those data records may be provided or exposed to the user device in an obfuscated form, which complies with data-privacy rules.

Cloud server 135 may be configured to execute intelligent functionality to process the data records stored in data registry 140. For example, executing intelligent functionality may include inputting at least a portion of the data records stored in data registry 140 into a trained machine-learning or artificial-intelligence models to generate outputs for further analysis. In some embodiments, the outputs can be used to extract patterns within the data records or to predict values or outcomes associated with data fields of the data records. Various embodiments of the intelligent functionality executed by cloud server 135 are described below.

In some embodiments, cloud server 135 is configured to enable a user device (e.g., operated by a doctor) to access the cloud-based application to transmit consult broadcasts to a set of destination devices. A consult broadcast may be a request for support or assistance regarding the treatment of a subject associated with a subject record. A destination device may be a user device operated by another user associated with another entity (e.g., a doctor at another medical center). If a destination device accepts the request for assistance associated with the consult broadcast, the cloud-based application may generate a condensed representation of the subject record that omits or obscures certain data fields of the subject record. The condensed representation may comply with data-privacy rules, and thus, the condensed representation of the subject record cannot be used to uniquely identify the subject associated by the subject record. The cloud-based application may transmit the condensed representation of the subject record to the destination device that accepted the request for assistance. The user operating the destination device may evaluate the condensed representation and communicate with the user device using a communication channel to discuss options for treating the subject. As an illustrative example, a physician may be treating a subject with a possible, probable or confirmed diagnosis of multiple sclerosis. The physician may seek additional advance or consult on how to treat the multiple sclerosis subject. The physician can cause a broadcast consult to be transmitted to physicians working at different hospitals. The various subject attributes of the subject record can be obfuscated and then transmitted to the other physician. The two physicians can then communicate regarding the obfuscated multiple sclerosis record during a communication session, such as a chatroom.

In some embodiments, cloud server 135 is configured to provide a treatment-plan definition interface to user devices. The treatment-plan definition interface enables user devices to define a treatment plan for a condition. For example, a treatment plan may be a workflow for treating a subject with the condition. A workflow may include one or more criteria for defining a population of subjects as having the condition. The workflow may also include a particular type of treatment for the condition. The cloud server 135 receives and stores treatment-plan definitions for a particular condition from each user device of a set of user devices. The cloud-based application may distribute a treatment plan for a given condition to a set of user devices. Two or more user devices of the set of user devices may be associated with different entities. Each of the two or more users devices may be provided with the option to integrate any portion or the entire treatment plan into a customer rule set. Cloud server 135 can monitor whether user devices integrate the shared treatment plan in full or integrate part of the treatment plan. The interactions between the user devices and the shared treatment plan can be used to determine whether to update the treatment plan or a rule created based on the treatment plan.

In some embodiments, cloud server 135 enables a user operating a user device to access the cloud-based application to determine a proposed treatment for a subject with a condition. The user device loads an interface associated with the cloud-based application. The interface enables the user operating the user device to select a subject record associated with a subject being treated by the user. The cloud-based application may evaluate other subject records to identify a previously-treated subject who is similar to the subject being treated by the user. The similarity between subjects, for example, may be determined using an array representation of the subject records. An array representation may be any numerical and/or categorical representation of the values of data fields of a subject record. For example, an array representation of a subject record may be a vector representation of the subject record in a domain space, such as in a Euclidean space. In some instances, multiple values in an array correspond to a single field. For example, a field value may be represented by multiple binary values generated via one-hot encoding. The cloud-based application may generate array representations for each subject record of a group of subject records. Similarity between two subject records may be represented by a distance between the array representations of the two subject records. Further, the cloud-based application may be configured to identify a subject who is a nearest neighbor to the subject record selected by the user device using the interface. The cloud-based application may identify treatments previously performed on the subject who is the nearest neighbor. The cloud-based application may avail on the interface the previously-performed treatments on the nearest neighbor for evaluation by the user operating the user device.

In some embodiments, cloud server 135 is configured to create queries that search a database of previously-treated subjects. Cloud server 135 may execute the queries and retrieve subject records that satisfy the constraints of the query. In presenting the query results, however, the cloud-based application may only present the subject record in full for subjects who have been or who are being treated by the user who created the query. The cloud-based application masks or otherwise obfuscates portions of subject records for subjects who are not being treated by the user creating the query. The masking or obfuscation of portions of subject records that are included in the query results enables the user to comply with data-privacy rules. In some embodiments, the query results (regardless of whether the query results are obfuscated or not) can be automatically evaluated for patterns or common attributes within the subject records.

In some embodiments, cloud server 135 embeds a chatbot into the cloud-based application. The chatbot is configured to automatically communicate with user devices. The chatbot can communicate with a user device in a communication session, in which messages are exchanged between the user device and the chatbot. A chatbot may be configured to select answers to questions received from user devices. The chatbot may select answers from a knowledge base accessible to the cloud-based application. When a user device transmits a question to the chatbot, and that chatbot does not have a preexisting answer stored in the knowledge base, then a different representation of the question for which there is a preexisting answer stored in the knowledge base. The user communicating with the chatbot can be prompted as to whether the answer provided by the chatbot is accurate or helpful.

### III.A. The Cloud-Based Application Enables User Devices To Broadcast Consult Requests to Other User Devices and Automatically Condenses Subject Records to Comply with Data-Privacy Rules

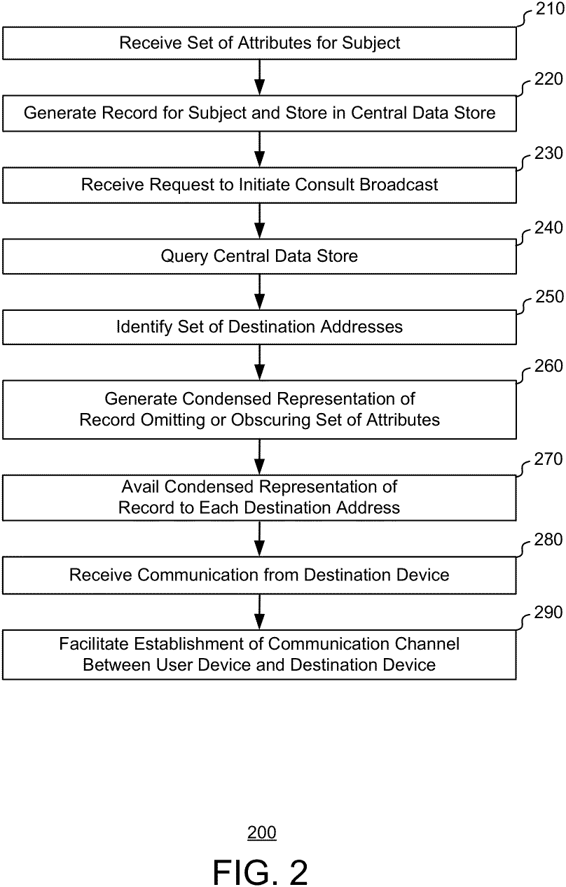

FIG. 2 is a flowchart illustrating process 200 performed by the cloud-based application to distribute condensed subject records to user devices in association with a consult broadcast requesting assistance with treating a subject. Process 200 may be performed by cloud server 135 to enable user devices associated with different entities (e.g., hospitals) to collaborate or consult regarding treatment for a subject, while complying with data-privacy rules.

Process 200 begins at block 210 where cloud server 135 receives a set of attributes from a user device. Each attribute of the set of attributes can represent any characteristic(s) of a subject (e.g., a patient). The set of attributes may be identified by a user using an interface provided by cloud server 135. For example, the set of attributes identify demographic information of the subject and a recent symptom experienced by the subject. Non-limiting examples of demographic information include age, sex, ethnicity, state or city of residence, income range, education level, or any other suitable information. Non-limiting examples of a recent symptom include a subject currently or recently (e.g., at a last visit, at intake, within 24 hours, within a week) experienced a particular symptom (e.g., difficulty breathing, fever above a threshold temperature, blood pressures above a threshold blood pressure, etc.).

At block 220, cloud server 135 generates a record for the subject. The record may be a data element including one or more data fields. The record indicates each of the set of attributes associated with the subject. The record may be stored at a central data store, such as data registry 140 or any other cloud-based database. At block 230, cloud server 135 receives a request, which was submitted by a user using the interface. The request may be to initiate a consult broadcast. For example, the user associated with an entity is a physician at a medical center treating a subject. The user can operate a user device to access the cloud-based application to broadcast a request for assistance with treating the subject. The broadcast may be transmitted to a set of other user devices associated with a different entity.

At block 240, cloud server 135 queries the central data store using the one or more recent symptoms included in the set of attributes associated with a subject. The query results include a set of other records. Each record of the set of other records is associated with another subject. At block 250, cloud server 135 identifies a set of destination addresses (e.g., other user devices associated with a different entity). Each destination address of the set of destination address is associated with a care provider for another subject associated with one or more other records of the set of other records identified at block 240. At block 260, cloud server 135 generates a condensed representation of the record for the subject. The condensed representation of the record omits, obscures, or obfuscates at least a portion of the record. The condensed representation of the record can be exchanged between external systems without violating data-privacy rules because the condensed representation of the record cannot be used to uniquely identify the subject associated with the record. Cloud server 135 can execute any masking or obfuscation techniques to generate the condensed representation of the record.

At block 270, cloud server 135 avails the condensed representation of the record with a connection input component to each destination address of the set of destination addresses. The connection input component may be a selectable element presented to each destination address. Non-limiting examples of the connection input component include a button, a link, an input element, and other suitable selectable elements. At block 280, cloud server 135 receives a communication from a destination device associated with a destination address. The communication includes an indication that the user operating the destination device selected the connection input component associated with the condensed representation of the record. At block 290, cloud server 135 facilitates the establishment of a communication channel between the user device and the destination device at which the connection input component was selected. The communication channel enables the user operating the user device (e.g., the physician treating the subject) to exchange messages or other data (e.g., a video feed) with the destination device associated with the destination address at which the connection input component was selected (e.g., a physician at another hospital who agreed to assist with the treatment of the subject).

In some embodiments, cloud server 135 is configured to automatically determine a location of the user device and a location of the destination device at which the connection input component was selected. Cloud server 135 can also compare the locations to determine whether to generate the condensed representation of the record. For example, at block 260, cloud server 135 may generate the condensed representation of the record because cloud server 135 determines that each destination address of the set of destination addresses is not collocated with the user device that initiated the consult broadcast. In this case, cloud server 135 may automatically determine to generate the condensed representation of the record to comply with data-privacy rules. As another example, if the set of destination addresses is associated with the same entity as the user device that initiated the consult broadcast, then cloud server 135 can transmit the record in full (e.g., without obfuscating a portion of the record) to a destination device associated with a destination address, while still complying with the data-privacy rules.

In some embodiments, cloud server 135 generates a plurality of other condensed record representations. Each of the plurality of other condensed record representations is associated with another subject. Cloud server 135 transmits the plurality of other condensed record representations to the user device; and receives, from the user device, a communication identifying selections of a subset of the plurality of other condensed record representations. Each of the set of destination addresses is represented by one of the condensed record representations. For example, generating a condensed record representation includes determining a jurisdiction of another subject associated with the condensed record representation, determining a data-privacy rule governing the exchange of subject records within the jurisdiction, and generated the condensed record representation to comply with the data-privacy rule. A first other condensed record representation of the plurality of other condensed record representations may include data of a particular type. A second other condensed record representation of the plurality of other condensed record representations may omit or obscure data of the particular type. For example, data of the particular type may be contact information, identifying information, such as name, social security number, and other suitable information that can be used to uniquely identify the other subject.

In some embodiments, querying the central data store using the one or more recent symptoms includes determining a score for each other record of a plurality of other records. The score can characterize a similarity between at least part of the other record and at least part of the record for the subject. The querying can further include defining the set of other records to be a subset of the plurality of other records associated for which the scores are above a threshold. The querying of the central data store can include using at least some of the demographic information to identify the set of other records. For example, one of the other records can include a data field containing an item of demographic information, such as age, sex, ethnicity, and so on. In some embodiments, the user device and the other device (e.g., the destination device associated with the destination address) are associated with different medical-care institutions.

### III.B. Updating Shareable Treatment-Plan Definitions Based on Aggregated User Integration

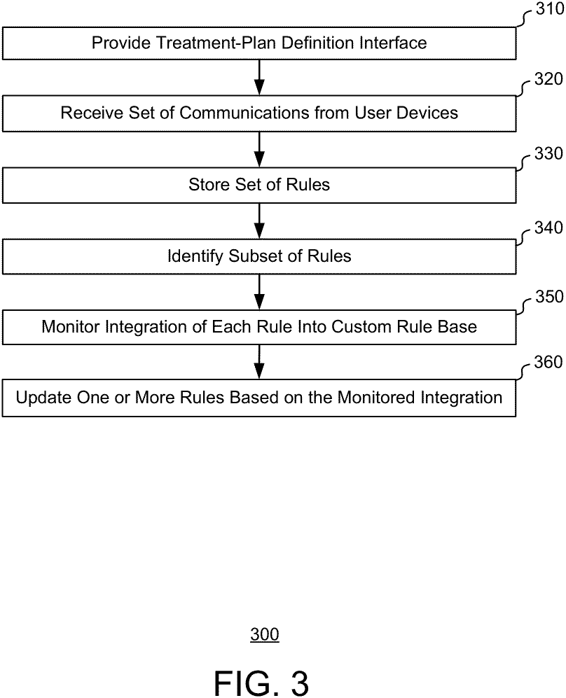

FIG. 3 is a flowchart illustrating process 300 for monitoring the user integration of treatment-plan definitions (e.g., decision trees or treatment workflows) and automatically updating the treatment-plan definitions based on a result of the monitoring. Process 300 may be performed by cloud server 135 to enable a user device to define a treatment plan for treating a population of subjects with a condition. The user device may distribute the treatment-plan definition to user devices connected to internal or external networks. The user devices receiving the treatment-plan definition can determine whether to integrate the treatment-plan definition into a custom rule base. The integration into the custom rule base can be monitored and used to automatically modify the treatment-plan definition.

At block 310, cloud server 135 stores interface data that causes a treatment-plan definition interface to be displayed when a user device loads the interface data. The treatment-plan definition interface is provided to each user device of a set of user devices when the user devices accesses cloud server 135 to navigate to the treatment-plan definition interface. In some embodiments, the treatment-plan definition interface enables a user to define a treatment plan for treating a population of subjects that have a condition (e.g., lymphoma).

At block 320, cloud server 135 receives a set of communications. Each communication of the set of communications is received from a user device of the set of user devices and was generated in response to an interaction between the user device and the treatment-plan definition interface. In some embodiments, the communication includes one or more criteria, for example, for defining a population of subject records. Each criteria may be represented by a variable type. A criterion may be a filter condition for filtering a pool of subject records. For example, a criteria for defining a population of subject records associated with subjects who may develop a lymphoma may include a filter condition of “abnormality in anaplastic lymphoma kinase (ALK)” AND “over 60 years old.” The communication may also include a particular type of treatment for the condition. The particular type of treatment may be associated with an action (e.g., undergo surgery) or non-action (e.g., reduce salt intake) that is proposed to treat the condition associated with the subjects represented by the population of subject records.

At block 330, cloud server 135 stores a set of rules in a central data store, such as data registry 140 or any other centralized server within cloud network 130. Each rule of the set of rules includes the one or more criteria and the particular treatment type included in the communication from a user device. As an illustrative example, a rule represents a treatment workflow for treating lymphoma in a subject. The rule includes the following criteria (e.g., the conditions following the “IF” statement) and a next action (e.g., the particular treatment type defined or selected by the user, and which follow the “THEN” statement): “IF ‘biopsy of lymph nodes indicates lymphoma cells are present’ AND ‘blood test reveals lymphoma cells present’ THEN ‘treat with chemotherapy’ AND ‘active surveillance.’”, Additionally, each rule of the set of rules is stored in association with an identifier corresponding to the user device from which the communication was received.

At block 340, cloud server 135 identifies a subset of the set of rules that are available across entities via the treatment-plan definition interface. A subset of rules may include the subset of the set of rules associated with a condition and that are distributed to external systems, such as other medical centers, for evaluation. For example, a rule can be selected for including in the subset of rules by evaluating a characteristic of the rule or the identifier associated with the rule. The characteristic of the rule can include a code or flag stored or appended to the stored rule. The code or flag indicates the rule is generally available to external systems (e.g., availed to entities).

At block 350, for each rule of the subset of rules identified at block 340, cloud server 135 monitors interactions with the rule. An interaction may include an external entity (e.g., external to the entity associated with the user who defined the treatment plan associated with the rule) integrating the rule into a custom rule base. For example, a user device associated with an external entity (e.g., a different hospital) evaluates the rule availed to the external entity. The evaluation includes determining whether the rule is suitable for integrating into a rule set defined by the external entity. The rule may be suitable when the user device associated with the external entity indicates that the treatment workflow that is defined using the rule is suitable to treat the condition corresponding to the rule. Continuing with the illustrative example above, the rule for treating lymphoma may be availed to an external medical center. A user associated with the external medical center determines that the rule for treating lymphoma is suitable for integrating into the rule set defined by the external medical center. Thus, after the rule is integrated into a custom rule base defined by the external medical center, other users associated with the external medical center will be able to execute the integrated rule by selecting the integrated rule from the custom rule base. Additionally, cloud server 135 monitors integration of the availed rule by detecting a signal generated or caused to be generated when the treatment-plan definition interface receives input corresponding to an integration of the rule into the custom rule base from the user device associated with the external entity.

As another illustrative example, the user device associated with the external entity uses the treatment-plan definition to integrate an interaction-specified modified version of the rule into the custom rule base. The interaction-specified modified version of the rule is a portion of the rule selected for integration into the custom rule base. Selecting a portion of the rule for integration includes selecting less than all criteria included in the rule for integration into the custom rule base. Continuing with the illustrative example above, the user device associated with the external entity selects the criteria of “IF ‘biopsy of lymph nodes indicates lymphoma cells are present’” for integration into the custom rule base, but the user device does not select the criteria of “blood test reveals lymphoma cells present” for integration into the custom rule base. Thus, the interaction-specific modified version of the rule integrated into the custom rule base is “IF ‘biopsy of lymph nodes indicates lymphoma cells are present’ THEN ‘treat with chemotherapy’ AND ‘active surveillance.’” The criteria of “blood test reveals lymphoma cells present” is removed from the rule to create the interaction-specified modified version of the rule, which is integrated into the custom rule base.

At block 360, cloud server 135 may detect that the interaction-specified modified version of the rule was integrated into the custom rule base defined by the external entity. Once detected, cloud server 135 may update the rule stored at the central data store of cloud network 130. The rule may be updated based on the monitored interaction(s). The term “based on” in this example corresponds to “after evaluating” or “using a result of an evaluation of” the monitored interaction(s). For example, cloud server 135 detects that the user device associated with the external entity integrated the interaction-specified modified version of the rule. In response to detecting the interaction-specified modified version of the rule, cloud server 135 may update the rule stored in the central data store from the existing rule to the interaction-specified modified version of the rule.

In some embodiments, cloud server 135 updates the rule by generating an updated version that is to be availed across external entities. Another original version may remain un-updated and is availed to a user associated with the user device from which the one or more communications that identified the criteria and particular type of treatment was received. For example, cloud server 135 updates the rule stored at the central data store, but cloud server 135 does not update another rule of the set of rules stored at the central data store.

In some embodiments, cloud server 135 may update the rule when an update condition has been satisfied. An update condition may be a threshold value. For example, the threshold value may be a number or percentage of external entities that have integrated a modified version of the rule into their custom rule bases. As another example, the update condition may be determined using an output of a trained machine-learning model. To illustrate, cloud server 135 may input the detected signals received from external entities into a multi-armed bandit model that automatically determines whether and when to avail the rule and/or whether and when to avail an updated version of the rule. The detected signals indicate whether an external entity integrated the rule into its custom rule base or whether the external entity integrated an interaction-specified modified version of the rule.

In some embodiments, cloud server 135 identifies multiple rules of the set of rules that include criteria corresponding to the same variable type and that identify same or similar types of treatment. A variable type may be a value or variable used as the condition of a criteria. The variable type of a criterion of a rule may also be any value of a condition that constrains the population of subjects to a sub-group. For example, the variable type of a rule that defines a population of pregnant women is “IF ‘subject is pregnant.’” Cloud server 135 determines a new rule that is a condensed representation of the multiple rules, when the new rule is generally availed across entities.

In some embodiments, cloud server 135 provides another interface configured to receive a set of attributes of a subject. For example, a user operating a user device to access the other interface and select a subject record that includes a set of attributes using the other interface. The selection of the subject record may cause the cloud server 135 to receive the set of attributes of the subject. Cloud server 135 identifies (e.g., determines) a particular rule for which the criteria are satisfied based on the set of attributes of the subject. For example, the evaluates the set of attributes of the subject record against the criteria of the rules stored in the central data store. To illustrate, if the set of attributes includes a data field containing the value “pregnant,” and if a rule includes a single criteria of “IF ‘subject is pregnant,” then cloud server 135 identifies this rule. Cloud server 135 updates the other interface to present the particular rule and each particular type of treatment associated with the particular rule.

In some embodiments, a criterion of a rule is a variable type that relates to a particular demographic variable and/or a particular symptom-type variable. Non-limiting examples of a demographic variable include any item of information that characterizes a demographic of the subject, such as age, sex, ethnicity, race, income level, education level, location, and other suitable items of demographic information. Non-limiting examples of a symptom-type variable indicate whether a subject currently or recently (e.g., at a last visit, at intake, within 24 hours, within a week) experienced a particular symptom (e.g., difficulty breathing, fainting, fever above a threshold temperature, blood pressures above a threshold blood pressure, etc.).

In some embodiments, cloud server 135 monitors data in a registry of subject records, such as the subject records stored in data registry 140. Cloud server 135 monitors the data in the registry of subject records for each rule of the subset of rules (identified at block 340). Cloud server 135 identifies a set of subjects for which the criteria of the rule were satisfied, and for which the particular treatment was previously prescribed to the subject. Cloud server 135 identifies, for each of the set of subjects, a reported state of the subject as indicated from or using assessment or testing. For example, the reported state is any information characterizing a state of the subject in an aspect, such as whether the subject has been discharged, whether the subject is alive, measurements of the subject’s blood pressure, the number of times the subject wakes up during a sleep stage, and other suitable states. Cloud server 135 determines an estimated responsiveness metric of the set of subjects to the particular treatment based on the reported states. For example, if the particular treatment of a rule is to prescribe a medication, the estimated responsiveness metric is a representation of the extent to which the medication addressed a symptom or condition experienced by the subject. As a non-limiting example, the estimated responsiveness metric of the set of subjects may be an average, weighted average, or any summation of a score assigned to each subject of the set of subjects. The score can represent or measure the effectiveness of the subject’s responsiveness to the treatment. Cloud server 135 can cause the subset of the set of rules and the estimated responsiveness metrics of the set of subjects to be displayed or otherwise presented in the treatment-plan definition interface.

### III.C. Presenting Treatment Recommendations with Associated Efficacy Using Treatments Prescribed to Similar Subjects

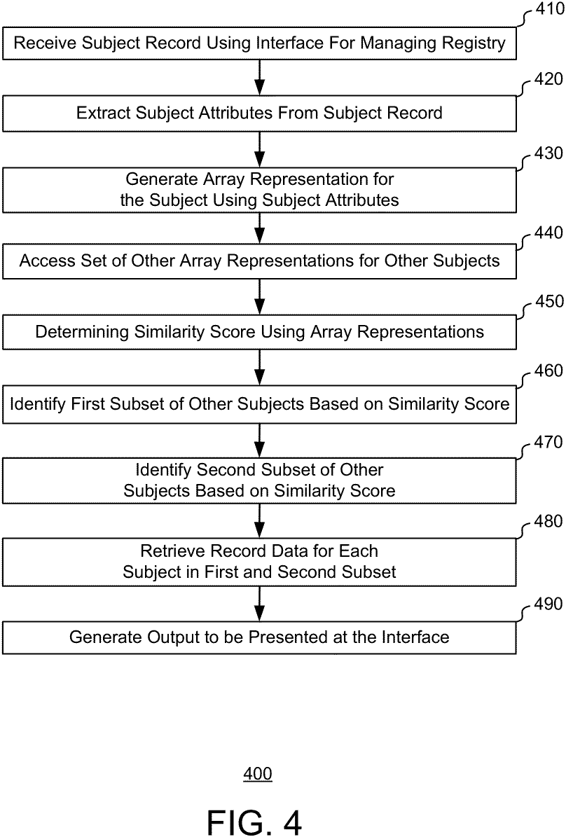

FIG. 4 is a flowchart illustrating process 400 for recommending treatments for a subject. Process 400 can be performed by cloud server 135 to display to a user device associated with a medical entity recommended treatments for a subject and the efficacy of each recommended treatment. The recommended treatments can be identified using a result of evaluating efficacies of treatments previously prescribed to similar subjects.

At block 410, cloud server 135 receives input corresponding to a subject record that characterizes aspects of a subject. The input is received from a user device associated with an entity. Further, the input is received in response to the user device selecting or otherwise identifying the subject record using an interface associated with an instance of a platform configured to manage a registry of subject records. User devices may access the interface by loading interface data stored at a web server (not shown) connected within cloud network 130. The web server may be included or executed on cloud server 135.

At block 420, cloud server 135 extracts a set of subject attributes from the subject record received at block 410. A subject attribute characterizes an aspect of the subject. Non-limiting examples of subject attributes include any information found in an electronic health record, any demographic information, an age, a sex, an ethnicity, a recent or historical symptom, a condition, a severity of the condition, and any other suitable information that characterizes the subject.

At block 430, cloud server 135 generates an array representation of the subject record using the set of subject attributes. For example, the array representation is a vector representation of the values included in the subject record. The vector representation may be a vector in a domain space, such as a Euclidean space. The array representation, however, can be any numerical representation of a value of a data field of the subject record. In some embodiments, cloud server 135 can perform feature decomposition techniques, such as singular value decomposition (SVD), to generate the values representing the set of subject attributes of the array representation of the subject record.

At block 440, cloud server 135 accesses a set of other array representations characterizing multiple other subjects. An array representation included in the set of other array representations may be a vector representation of a subject record that characterizes another subject (e.g., one of the multiple other subjects).

At block 450, cloud server 135 determines a similarity score representing a similarity between the array representation representing the subject and the array representation of each of the other subjects. For example, the similarity score is calculated using a function of a distance (in the domain space) between the array representation representing the subject and the array representation representing the other subject. To illustrate and as only a non-limiting example, the similarity score may be calculated using a range of “0” to “1,” with “0” representing a distance beyond a defined threshold and “1” representing that the array representations have no distance between them.

At block 460, cloud server 135 identifies a first subset of the multiple other subjects. Subjects may be included in the first subset when the similarity score associated with a subject is above a predetermined absolute or relative threshold. Similarly, at block 470, cloud server identifies a second subset of the multiple other subjects. However, subjects may be included in the second subset when the similarity score of this subject is within a predetermine range.

At block 480, cloud server 135 retrieves record data for each subject in the first subset and in the second subset of the multiple other subjects. The record data include the attributes that are included in a record characterizing a subject. For example, the record data identifies a treatment received by the subject and the subject’s responsiveness to the treatment. The responsiveness to the treatment may be represented by text (e.g., “subject responded positively to treatment”) or a score indicating an extent to which the subject responded positively or negatively to the treatment (e.g., a score from “0” to “1” with “0” indicating a negative responsiveness and “1” indicating a positive responsiveness).

At block 490, cloud server 135 generates an output to be presented at the interface on the user device. The output may indicate, for example, the treatments received by the other subjects in the first and second subsets, the treatment responsiveness of subjects in the first and second subsets, and the differences between the subject attributes of subjects in the second subset and subject attributes of the subject.

In some embodiments, cloud server 135 determines that the subject and one of the subjects from the first or second subset are being treated or were treated by the same medical entities. Cloud server 135 determines that the subject and another subject of the first or second subset are being treated or were treated by different medical entities. Cloud server 135 may avail differentially obfuscated versions of records of the subjects via the interface. As a technical advantage, the cloud-based application can automatically provide differently obfuscated versions of records to entities based on varying constraints imposed on data sharing by the data-privacy rules of different jurisdictions. In some embodiments, cloud server 135 identifies the first subset and the second subset using a nearest-neighbor learning technique.

### III.D. Automatically Obfuscating Query Results from External Entities

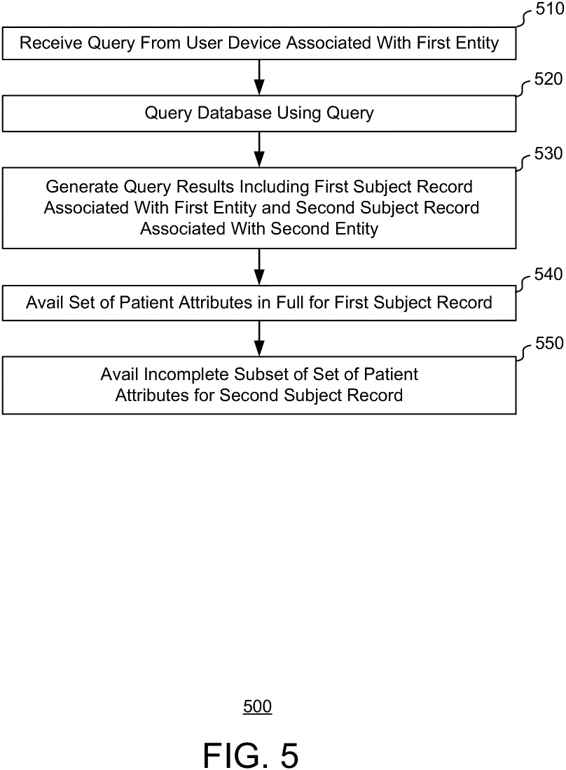

FIG. 5 is a flowchart illustrating process 500 for obfuscating query results to comply with data-privacy rules. Process 500 may be performed by cloud server 135 as an executing rule that ensures data sharing of subject records with external entities complies with data-privacy rules. The cloud-based application may enable a user device to query data registry 140 for subject records that satisfy a query constraint. The query results, however, may include data records originating from external entities. Thus, process 500 enables cloud server 135 to provide user devices with additional information on treatments from external entities, while complying with data-privacy rules.

At block 510, cloud server 135 receives a query from a user device associated with a first entity. For example, the first entity is a medical center associated with a first set of subject records. The query may include a set of symptoms associated with a medical condition or any other information constraining a query search of data registry 140.

At block 520, cloud server 135 queries a database using the query received from the user device. At block 530, cloud server 135 generates a data set of query results that correspond to the set of symptoms and are associated with the medical conditions. For example, the user device transmits a query for subject records of subjects who have been diagnosed with lymphoma. The query results include at least one subject record from the first set of subject records (which originate or were created at the first entity) and at least one subject record from a second set of subject records associated with a second entity (e.g., a medical center different from the first entity). Each of the subject record from the first set of subject records and the subject record from the second set of subject records may include a set of subject attributes. A subject attribute can characterize any aspect of a subject.

At block 540, cloud server 135 presents (e.g., avails or otherwise makes available) to the user device the set of subject attributes in full for subject records included in the first set of subject records because these records originate from the first entity. Presenting a subject record in full includes making the set of attributes included in a subject record available to the user device for evaluation or interaction using the interface. At block 550, cloud server 135 also or alternatively avails to the user device an incomplete subset of the set of subject attributes for each subject record included in the second set of subject records. Providing an incomplete subset of the set of subject attribute provides anonymity to subjects because the incomplete subset of subject attributes cannot be used to uniquely identify a subject. For example, providing an incomplete subset may include available four of 10 subject attributes to anonymize the subject associated with the 10 subject attributes. In some embodiments, at block 550, cloud server 135 avails an obfuscated set of subject attributes for each subject record included in the second subject. Obfuscating the set of attributes include reducing the granularity of information provided. For example, instead of availing the subject attribute of a subject’s address, the obfuscated attribute may be a zip code or a state in which the subject lives. Whether an incomplete subject or an obfuscated subset is availed, cloud server 135 anonymizes a subject associated with the subject record.

### III.E. Chatbot Integration with Self-Learning Knowledge Base

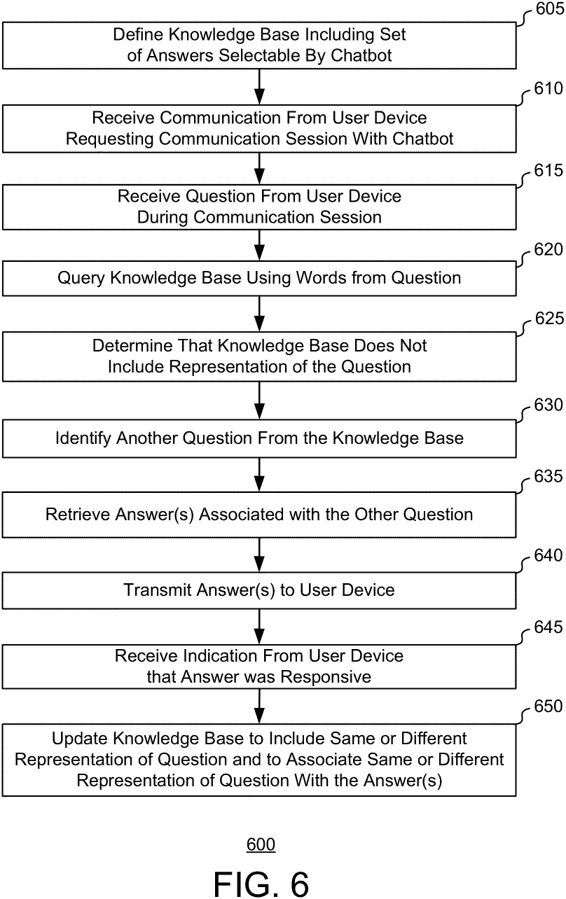

FIG. 6 is a flowchart illustrating process 600 for communicating with users using bot scripts, such as a chatbot. Process 600 may be performed by cloud server 135 for automatically linking new questions provided by users to existing questions in a knowledge base to provide a response to the new question. A chatbot may be configured to provide answers to questions associated with a condition.

At block 605, cloud server 135 defines a knowledge base, which includes a set of answers. The knowledge base may be a data structure stored in memory. The data structure stores text representing the set of answers to defined questions. Each answer may be selectable by a chatbot in response to a question received from a user device during a communication session. The knowledge base may be automatically defined (e.g., by retrieving text from a data source and parsing through the text using natural language processing techniques) or user defined (e.g., by a researcher or physician).

At block 610, cloud server 135 receives a communication from a particular user device. The communication corresponds to a request to initiate a communication session with a particular chatbot. For example, a physician or subject may operate a user device to communicate with a chatbot in a chat session. Cloud server 135 (or a module stored within cloud server 135) may manage or facilitate the establishment of communication sessions between user devices and chatbots. At block 615, cloud server 135 receives a particular question from the particular user device during the communication session. The question can be a string of text that is processed using natural language processing techniques.

At block 620, cloud server 135 queries the knowledge base using at least some words extracted from the particular question. The words may be extracted from the string of text representing the particular question using natural language processing techniques. At block 625, cloud server 135 determines that the knowledge base does not include a representation of the particular question. In this case, the question received may be newly posed to a chatbot. At block 630, cloud server 135 identifies another question representation from the knowledge base. Cloud server 135 may identify another question representation by comparing the question received from the user device to the other question representations stored in the knowledge base. If a similarity is determined, for example, based on an analysis of the question representations using natural language processing techniques, then cloud server 135 identifies the other question representation.

At block 635, cloud server 135 retrieves an answer of the set of answers associated, in the knowledge base, with the other question representation. At block 640, the answer retrieved at block 635 is transmitted to the particular user device as an answer to the question received, even though the knowledge based did not include a representation of the question received. At block 645, cloud server 135 receives an indication from the particular user device. For example, the indication may be received in response to the user device indicating that the answer provided by the chatbot was responsive to the particular question.

At block 650, cloud server 135 updates the knowledge base to include the representation of the particular question or different representation of the particular question. For example, storing a representation of a question includes storing keywords included in the question in a data structure. Cloud server 135 may also associate the same or different representation of the particular question with the more answer transmitted to the particular user device.

In some embodiments, cloud server 135 accesses a subject record associated with the particular user device. Cloud server 135 determines a plurality of answers to the particular question. Cloud server 135 then selects an answer from the set of answers. The selection of the answer, however, is based at least in part on one or more values included in the subject record associated with the particular user device. For example, a value included in the subject recode may represent a symptom recently experienced by the subject. The chatbot may select an answer that is dependent on the symptom recently experienced by the subject.

### III.F. Module for Predicting Responses to Multiple Sclerosis Treatments and Monitoring Multiple-Sclerosis Progression

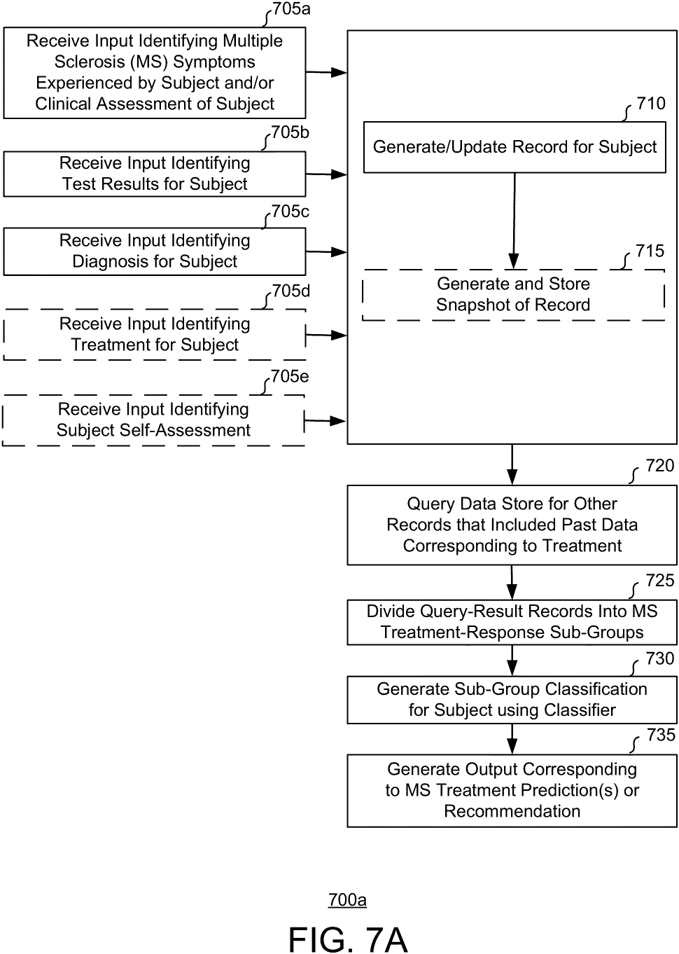

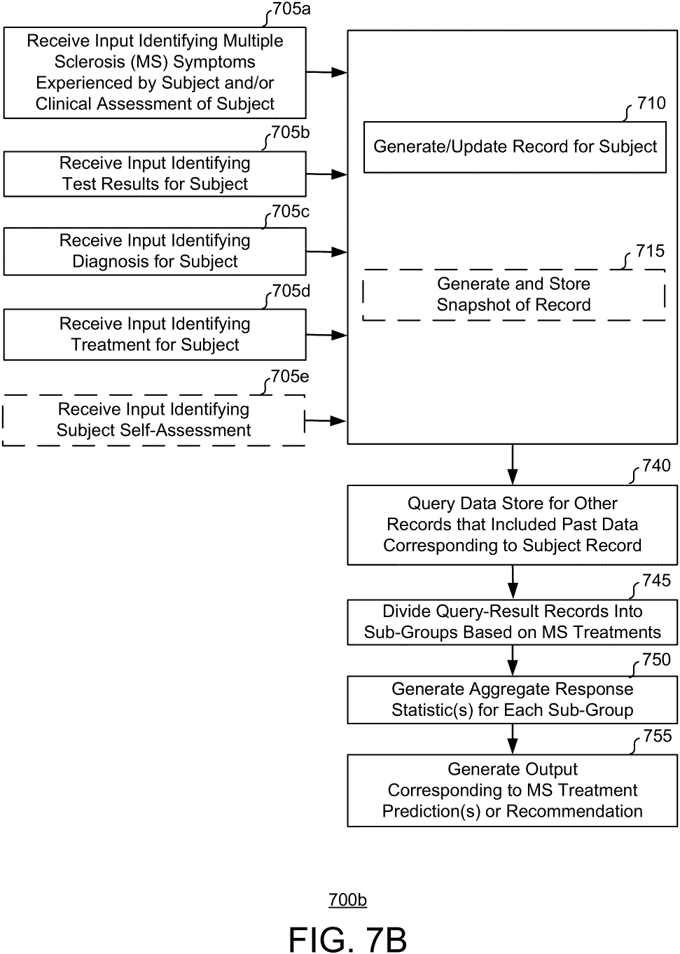

FIGS. 7A and 7B depict flowcharts illustrating example processes 700a and 700b for building and using snapshot data store representing dynamic and distributed-source data to characterize subpopulations and generate subject-specific predictions. Process 700a (depicted in FIG. 7A) begins at blocks 705a-705e, with the receipt of input identifying information pertaining to a subject with possible, probable or confirmed multiple sclerosis. The inputs received at the various blocks may be received at different times, from different computing systems and in association with different users. The inputs can be received via interfaces (e.g., web-based or app-based interfaces) generated and/or managed by a cloud-based application system.

At block 705a, input is received that identifies one or more symptoms experienced by a subject and/or a clinical assessment of the subject. The symptoms may include one or more multiple sclerosis symptoms as identified herein, one or more neurological systems, and/or one or more symptoms associated with one or more functional systems. The clinical assessment may include (for example) assessing a mobility of a subject, a disability of a subject (e.g., in accordance with a defined disability scale, such as EDSS), an identification of a time required to perform a given task (e.g., to walk a particular distance, perform a peg test, etc.), an identification of an accuracy of a task performance (e.g., a memory task, a cognitive task, etc.), and so on. The input received at block 705a may be from (for example) a care provider, physician, neurologist, nurse practitioner, nurse, physician’s office, hospital and/or subject.

At block 705b, input is received that identifies one or more test results associated with the subject. A test result can include a result of one or more tests used to diagnosis and/or assess multiple sclerosis, such as a test identified in Section II (e.g., an MRI, CSF analysis, visually evoked potential test and/or a blood test). The input received at block 705b may be from (for example) a laboratory technician, a radiologist, a laboratory and/or an imaging center.

At block 705c, input is received that identifies a possible, probable or confirmed diagnosis for a subject. The diagnosis may include a neurological disease, multiple sclerosis or a sub-type of multiple sclerosis (e.g., one identified in Section II). The input received at block 705c may be from (for example) a neurologist, physician, nurse, nurse practitioner, doctor’s office or hospital.

At optional block 705d, input is received that identifies a treatment for a subject. The treatment may be one that has been or is being prescribed for the subject or one that is being considered for the subject. The treatment may include a multiple sclerosis treatment, such as one described herein (e.g., in Section II).

At optional block 705e, input is received that identifies a subject’s self-assessment. The subject’s self-assessment can correspond to well-being, symptom presentation, state of mind, activity level, social engagement, treatment objectives, etc. In some instances, at least some of the input received at block 705e is responsive to a quality-of-life survey, such as the Multiple-Sclerosis Quality of Life-54 questionnaire.

It will be appreciated that inputs received at a given box may be received at multiple times. For examples, symptom identification at box 705a may be reported multiple times (e.g., reflecting a current state of symptoms).

The inputs are used to generate and/or update a record for a particular subject at block 710, using a cloud-based application. In some instances, in order to enter the input, a user may need to either first locate an existing record or create a new one. In some instances, input is automatically accepted, and the cloud system determines whether subject-identifying information sufficiently matches a record (and if not, a new record can be generated). Data conveyed by each input may be associated with a timestamp and source. In some instances, the data is processed (e.g., to facilitate storing the data in a more structured and/or standard form). For example, specific numbers may be converted into ranges, MRI images may be processed via image processing to generate statistics, etc.

At optional block 715, a record snapshot is generated. Record snapshots may be generated at defined times or defined time intervals or in response to particular types of input. For example, a new snapshot may be generated upon detecting a new diagnosis, treatment, symptom and/or MRI results. A snapshot may include a value for each of a set of fields. The set of fields may characterize a corresponding subject (e.g., via demographic information, medical history and/or behavioral patterns), a diagnosis, a diagnosis history, a current treatment, a treatment history (e.g., which treatments were received, for which durations and/or during which time periods), current symptoms, symptom history and/or recent self-assessments. Values for all of these fields are potentially not all concurrently provided. Thus, the cloud-based application may identify most recent values for fields not provided in a snapshot-triggering input. In some instances, if a most recent value is sufficiently old, it may be omitted from or flagged in the snapshot.

At block 720, the data store is queried for other records that include past data corresponding to a particular treatment. The particular treatment may be one that is being received by the subject or one that is being considered for the subject. In some instances, the query includes a temporal constraint, such as requiring that the particular treatment was initiated at least a year ago (such that subsequent data is likely available). In some instances, the query further includes one or more attributes of the subject (e.g., age, geography, disease type, duration from initial multiple-sclerosis diagnosis, disability, etc.). In these instances, the query may be performed to identify records indicating the corresponding subjects had those attributes at a specific time (or time period) relative to when the particular treatment was initiated. For example, the attributes may be those associated with the subject when or shortly before the particular treatment was initiated. As another example, the specific time may be the length of time that the given subject (for which input was provided at blocks 705a-705e) has been receiving the particular treatment. The query may thus be performed using snapshots to facilitate the temporal dependency restriction.

Notably, while the query may be performed with a first time constraint (e.g., to search for records and/or snapshots associating particular attributes with a time at which a particular treatment began), data returned by the query may be associated with a different time period (e.g., approximately one year after initiation of the treatment, the first two years of the particular treatment’s use, a time between initiation of the particular treatment and termination of the particular treatment). Thus, for example, a query constraint may indicate that a subject is to have fewer than 5 lesions when initiating a treatment, while a result of the query may indicate how the number of lesions changed over the time during which the treatment was being used.

At block 725, the records provided in response to the query are divided into two or more sub-groups. Each sub-group may correspond to a different type of response to the treatment. For example, sub-groups may differ with regard to how long subjects remained on a treatment (e.g., with longer durations suggesting higher efficacy), with regard to MRI progression, with regard to disability progression, with regard to symptom incidence, with regard to progress across MS sub-types (e.g., from relapse-remitting to secondary progressive) and/or combinations thereof. This sub-group division may be determinable based on data points collected from the records within time windows corresponding to treatment periods (e.g., within a defined time period from an initiation of the treatment or between the initiation of the treatment until another treatment is used).

At block 730, a classifier is used to assign the subject to one of the sub-groups based on characteristics of the subject. The classifier may include (for example) a clustering classifier, neural network (e.g., perceptron, decision tree, random forest, logistic regression, linear regression, nearest neighbor, naive bayes), component-analysis classifier, etc. The classifier may learn to identify one or more features associated with each sub-group and to generate a similarity metric that assesses how similar a profile of the subject is to a given class (e.g., versus one or more other sub-groups). The classification may indicate a prediction as to whether and/or a degree to which the subject will respond to the treatment.

At block 735, an output is generated that corresponds to a treatment prediction or recommendation based on the classification generated at block 730. For example, the output may identify a binary prediction as to whether the subject will respond to the treatment, a probability that the subject will respond to the treatment, a predicted progression of the subject on the treatment over a time period, a predicted change in MRI statistics for the subject on the treatment over a time period, a probability of progression to another disease sub-type, etc. The output may be presented at and/or transmitted to (for example) a device associated with a care provider, neurologist, physician or subject. In some instances, process 700a is repeated for each of multiple treatment options, such that a treatment option associated with a most favorable predicted outcome for a subject may be identified.

In some instances, part or all of blocks 740-755 are performed automatically. For example, the query at block 720 may be performed at routine time intervals, in response to receiving a particular type of input about a subject (e.g., identification of MRI or disability progression, identification of a new treatment being used or considered for the subject, or identification of a new diagnosis of an MS sub-type for the subject). The query may be structured to include a constraint identifying a particular treatment being used or considered for the subject as reflected in the subject’s record. The query may request particular predefined field values (e.g., corresponding to MRI results, relapse detections, disability assessments, adverse events, subsequent treatment changes, subject attributes, diagnoses and/or prior medications used) for those records satisfying the constraint. The requested particular predefined field values may be associated with a predefined time period relative to initiation of the particular treatment (e.g., first year of the treatment, first two years of the treatment or duration of the treatment).

As another example, an automated technique or a predefined criteria may be used to divide the records at block 725. The automated technique may include (for example) a clustering algorithm applied to MRI-change data, disability-change data, or length-of-treatment use data. The predefined criteria may separate records based on (for example) a threshold number of new lesions detected during a treatment period, a change in disability score detected during a treatment period and/or a number of relapses detected of a treatment period.

One benefit of automated processing is that it may shield record data from a user that receives the output generated at block 735. That is, a user or user device may lack access to specific values from records retrieved by the query at block 720. This can facilitate data privacy while continuing to capitalize on big data.

In some instances, part or all of blocks 720-735 are performed in response to input (e.g., from a same or different user as one that provided an input received at any of blocks 705a-705e). For example, the user input may select fields for which record values are to be retrieved via the query (at block 720) upon detecting constraint satisfaction and/or the user input may identify a criteria for separating the query-result records into sub-groups at block 725. In instances in which a user is more actively controlling blocks 720-725, data presentations and/or visualizations may be abstracted, obfuscated and/or generalized so as to protect data privacy. For example, distributions and/or statistics of values of various fields may be presented (e.g., representing records retrieved by the query) rather than presenting particular field values. The distributions and/or statistics may even be presented for individual fields rather than presenting multi-variate distributes and/or statistics to again veer away from presenting identifying or personal information.

Process 700b, depicted in FIG. 7B, illustrates another technique for using subject records to inform treatment predictions or recommendations. Blocks 705a-705e, 710 and 715 depicted in FIG. 7B can correspond to the similarly numbered blocks depicted in FIG. 7A. At block 740, the data store is queried for corresponding records that include past data corresponding to the subject record. For example, the query may identify one or more symptoms, test results, diagnoses (e.g., of a multiple-sclerosis subtype), treatments and/or self-assessments that are associated with the subject for which input was received at blocks 705a-705e. In some instances, the query includes one or more values received via input at blocks 705a-705e or a processed version thereof. For example, input at block 705a may identify a given subject as being 27 years old, living at a particular address is Des Moines, Iowa, with 8 T2-scan MRI lesions, while a query constraint may specify that snapshots of interest are to be associated with subjects between 21 and 30 years old, living in the United States having 6-10 T2 lesions.

In some instances, for each of a set of other-subject snapshots, a similarity score is generated using at least part of the snapshot or record associated with the given subject for which input was received at blocks 705a-705e and based on at least part of the other-subject snapshot. Different fields may be associated with different weights to generate the score. Block 740 may then include identifying other-subject snapshots for which the score was above a predefined threshold (e.g., within a top percentile or above a particular value).

As described above with respect to block 720 from FIG. 7A: while the query performed at block 740 in FIG. 7B may be performed with a first time constraint (e.g., to search for records and/or snapshots associating particular attributes with a time at which a particular treatment began), data returned by the query may be associated with a different time period (e.g., approximately one year after initiation of the treatment, the first two years of the particular treatment’s use, a time between initiation of the particular treatment and termination of the particular treatment). Thus, in some instances, constraints of the query may apply to individual snapshots (e.g., such that an individual snapshot is to satisfy all constraints), whereas record information that is retrieved may then correspond to other snapshots and/or other parts of the records (associated with same subjects for which the constraints were satisfied).

At block 745, the query-result records are divided into sub-groups based on treatments. For example, each query result may correspond to a portion of a corresponding record that identifies data points corresponding to a time when a given treatment was initiated, being used and/or terminated. Thus, potentially, for a given subject, multiple query results are identified – each corresponding to a different treatment. The sub-division at block 745 may include identifying unique MS treatments and dividing query results accordingly. In some instances, multiple treatments are associated with an individual group (e.g., when the multiple treatments are associated with a same or similar mechanism of action). For example, interferon-beta and glatiramer acetate may be grouped together. As another example, dimethyl fumerate, monomethyl fumerate and diroximel fumarate can be grouped together. As another example, ocrelizumab, ofatumumab, ublituximab and rituximab can be grouped together.

At block 750, one or more response statistics are generated for each sub-group. The response statistics may be generated based on (for example) clinical, MRI, symptomatic, treatment decisions, wellness indices, adverse events, and/or relapse data associated with a time period during which a treatment was received. The response statistics may reflect absolute values (e.g., an absolute lesion load and/or absolute disability score) and/or a change in values across a treatment period (e.g., change in lesion load since treatment initiation and/or a change in disability score since treatment initiation). The response statistic(s) can be based on (for example) a number of T2 lesions, number of enhancing lesions, cumulative number of enhancing lesions, a lesion load, atrophy metric, number of relapses, disability score, wellness index, a length of time that a subject remained on the treatment, and/or one or more other MS-pertinent variables disclosed herein. In some instances, the response statistic is based on multiple metrics. For example, the response statistic may include a binary value indicating whether a subject experienced any new lesions, any relapses or any disability progression over a period of time. The response statistic(s) can include (for example) a univariate distribution, multivariate distribution, mean, median, mode, skew, range, maximum, minimum, and/or standard deviation. The response statistic(s) can include a percentage of subjects for which a particular type of response (e.g., corresponding to not worsening) was observed).

At block 755, an output is generated that corresponds to one or more predictions as to how the subject associated with the record generated/updated at block 710 would respond to each of one or more treatments and/or identifying a recommended treatment. A prediction as to how the subject would respond to a treatment may include a response statistic generated for a corresponding sub-group. For example, the prediction may identify a 44% chance of lesion load and disability remaining stable for at least two years if a given medication is taken over that time period. The recommended treatment may be associated with one or more most favorable response statistics relative to statistics associated with other treatments. The output may be transmitted to a user device and/or presented locally (e.g., to a care provider, physician, neurologist or subject). The output may include one, more or all of the response statistics generated at block 750, which may be identified in association with corresponding treatments.

As in FIG. 7A, part or all of the query and subsequent processing may be performed automatically or in response to user input. For example, a predefined rule may identify which subject attributes are to be used in the query (e.g., and how they are to be generalized), or user input may identify similar information. As another example, a predefined protocol may indicate what types of statistics are to be generated at block 750. As yet another example, an interface may present – for each of one or more types of response variables (e.g., new lesion count, new relapse count, disability progression, adverse-event count) – a distribution for each of the sub-groups (e.g., as overlaid lines or separate graphs). The user may then select a response variable of interest and define one or more statistics to be generated for each treatment type.

Thus, each of processes 700a and 700b depicted in FIGS. 7A and 7B can facilitate identifying a treatment using big-data processing. It will be appreciated that similar approaches may be used to generate a predicted prognosis (e.g., associated with use of a given treatment or irrespective of specific treatment).

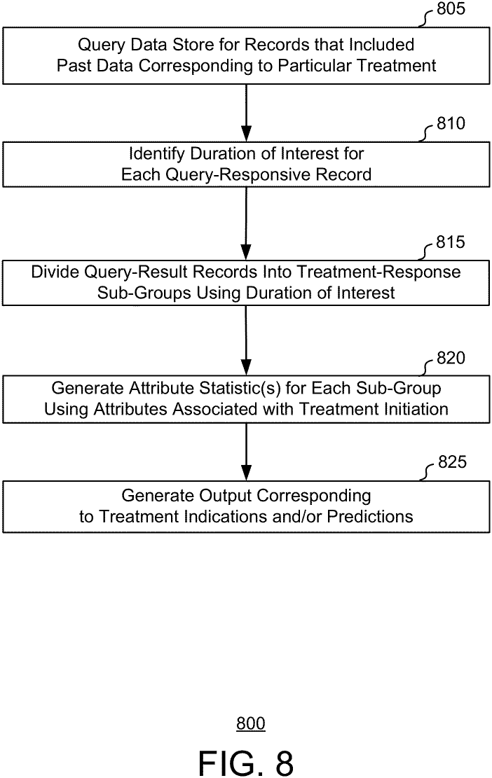

In some instances, the data store may be used to generate more general treatment predictions and/or indications (e.g., not necessarily tied to an individual subject). FIG. 8 depicts a flowchart illustrating an example process 800 for using a snapshot data store to generate high-level treatment-response predictions and/or indications. Process 800 begins at block 805 where a data store is queried for records that include an indication that a subject received a particular treatment. The query may specify that the particular treatment was to have been initiated at least by a particular date (e.g., over a year ago, over 2 years ago, over 5 years ago). In some instances, the query further specifies one or more other constraints, such a sub-type of multiple sclerosis that the subject was to have had when initiating the particular treatment. The data store queried at block 805 can include one that is generated based on one or more input types and/or variables disclosed herein. In some instances, the data store includes a set of snapshots associated with each individual subject to reflect estimated time-synchronized data points.

At block 810, a duration of interest is identified for each query-responsive record. The duration of interest may correspond to a period of time beginning at initiation of the particular treatment and over which one or more response metrics are to be assessed. In some instances, the duration of interest is the same across query-responsive records (e.g., one year, two years, five years). In some instances, the duration of interest is defined to be a period of time over which the particular treatment was used. In some instances, the duration of interest is a shorter of a period of use of the particular treatment and a specific time.

At block 815, the query-result records are divided into treatment-response sub-groups. The records may be divided in accordance with one or more techniques and/or based on one or more variables described with respect to block 725 in process 700a (as depicted in FIG. 7A). In some instances, the records are divided into two sub-groups (corresponding to progression and no progression; new lesions and no new lesions; new symptoms and no new symptoms; moderate-severe adverse events and no or minor adverse events; use of the treatment for at least a threshold amount of time and use of the treatment for less than the threshold amount of time). In some instances, the sub-group assignments depend on more than one variables. In some instances, there are more than two sub-groups.

The variable values that are used for the record division may have been extracted from record entries associated with the duration of interest identified in block 810. For example, if a duration of interest is identified as being one year: for each query-result record, the record may be searched for entries that represent treatment responsiveness (e.g., MRI data, clinical assessment, medication status) and that are associated with timestamps approximately one year out from treatment initiation. As another example, if a duration of interest is identified as a treatment duration, all snapshots associated with timestamps subsequent to treatment initiation and extending through a snapshot indicating a change in medication may be identified. Thus, if a subject began receiving a treatment in April 2013 and terminated the treatment in May 2016, the subject’s lesion count or disability in October 2017 may be (determining on the duration of interest) irrelevant for the sub-dividing of the records.

At block 820, one or more aggregate attribute statistics are generated for each sub-group. An aggregate statistic can characterize an attribute of subjects in a sub-group at a time at which the subjects began receiving the particular treatment. The aggregate attribute statistic(s) may correspond to (for example) a subject’s age, sex, latitude, state of residence, country of residence, sub-type of MS, medication history, lesion count, lesion load, disability score, mobility indicator, walking-aid use, co-morbidity status, functional systems affected by symptoms, and/or length of time since initial MS diagnosis – again, all considered at a time point at which treatment was initiated. The aggregate attribute statistic(s) can include a mean, median, mode, range, minimum, maximum, outlier, distribution, skew, etc.

At block 825, an output is generated corresponding to treatment indications and/or predictions. For example, an output can identify attribute characteristics (e.g., via one or more attribute statistics) associated with a sub-group for which the particular treatment was effective and can identify attribute characteristics associated with another sub-group for which the particular treatment was not effective. In some instances, characteristics of only a subset of a set of attributes assessed are represented in the output. For example, at block 820, for each of a set of attributes, a p-value (or other significance indicator) may be generated to indicate an extent to which the attributes are distinguishable across sub-groups. The output generated at block 825 can then include a sub-group-specific statistic for each attribute associated with a p-value that is below a threshold (or with another significance indicator indicating significant sub-group distinctions. In some instances, a multi-variate analysis is performed to identify attributes that are most predictive of sub-group assignments, and the output characterizes values of those attributes in one, more or all sub-groups.

In some instances, block 825 includes generating a new treatment indication. For example, the attribute statistics may indicate that the particular treatment is especially well-suited (e.g., as determined based on duration of use) when used by RRMS MS patients younger than 50 years old and having an EDSS score that is less than 3.5. As another example, the attribute statistics may indicate that the particular treatment is correlated with particular fast progression when used by RRMS patients having an EDSS score greater than 6.0. Subsequent analysis may then be performed to assess whether there is a significant difference between one of these subject populations when the particular treatment is being taken as compared to when one or more other treatments (or no treatment) is being received. If so, treatment strategies may be employed to determine whether to recommend or use the particular treatment for a given subject by determining whether the given subject’s attributes corresponds to a one of the populations.

In some instances, rather than dividing query-result records into sub-groups to assess how subject attributes correlate with treatment response, treatment responses may be identified as a non-binary numeric number. For example, a treatment response may include a number of weeks that a subject remained on the treatment, a number of new lesions detected over the course of two years while on the treatment, a change in a numerical well-being index over the first year of the treatment, etc. A model may then be trained to predict the treatment response based on attributes of the subject. For example, a regression model or feedforward neural network may be used. The trained model can subsequently process an input data set representing another subject’s attributes to predict how the subject would respond to the treatment. Learned parameters may also be assessed to determine which subject attributes are associated with high weights (e.g., indicating relatively that they are relatively influential in generating output predictions).

### IV. Example

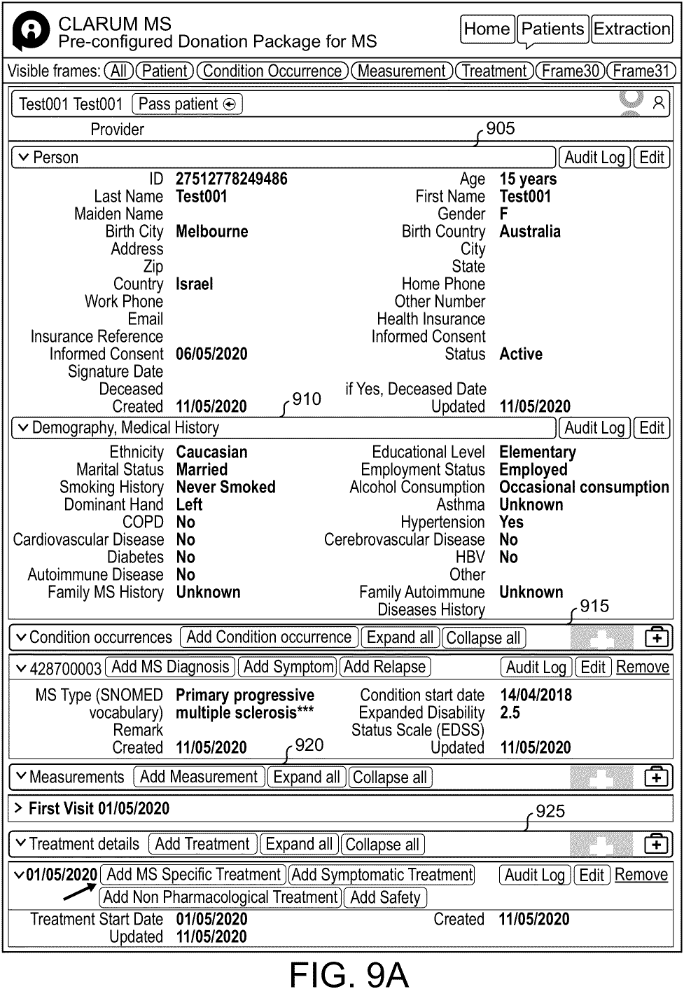

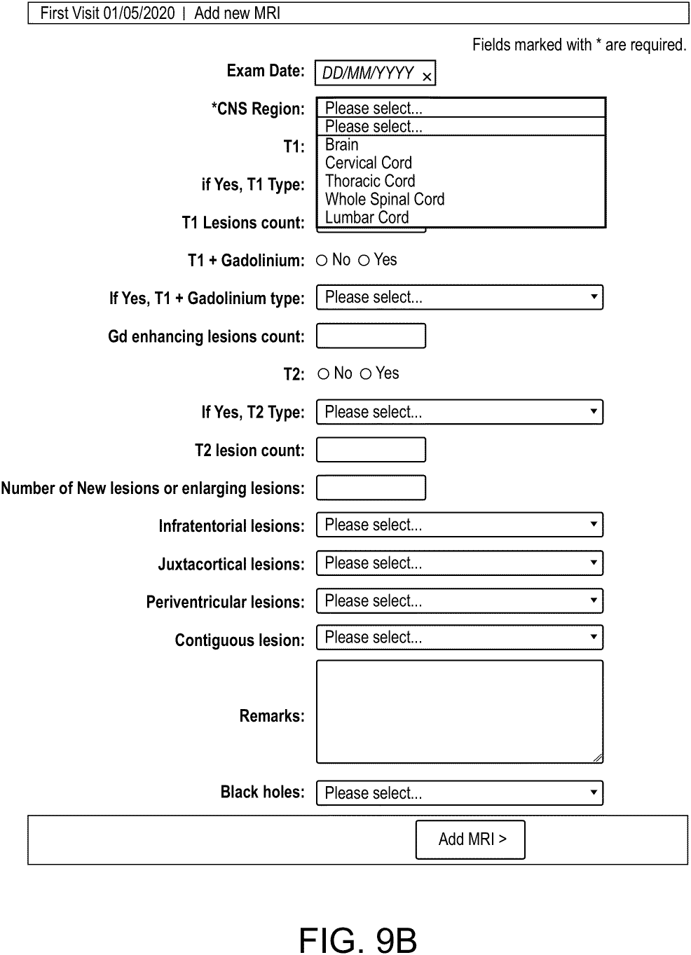

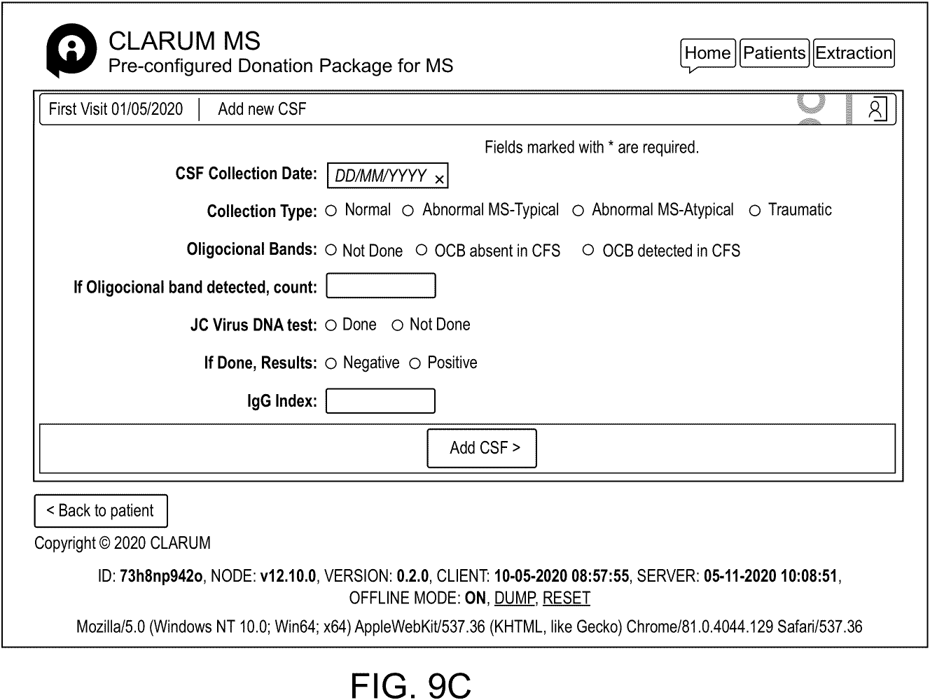

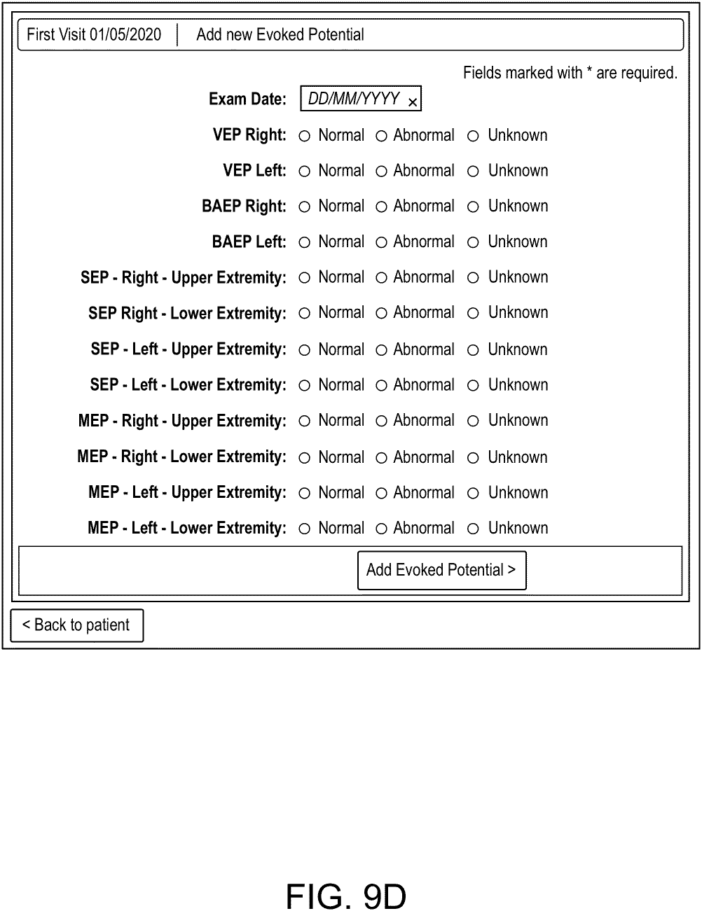

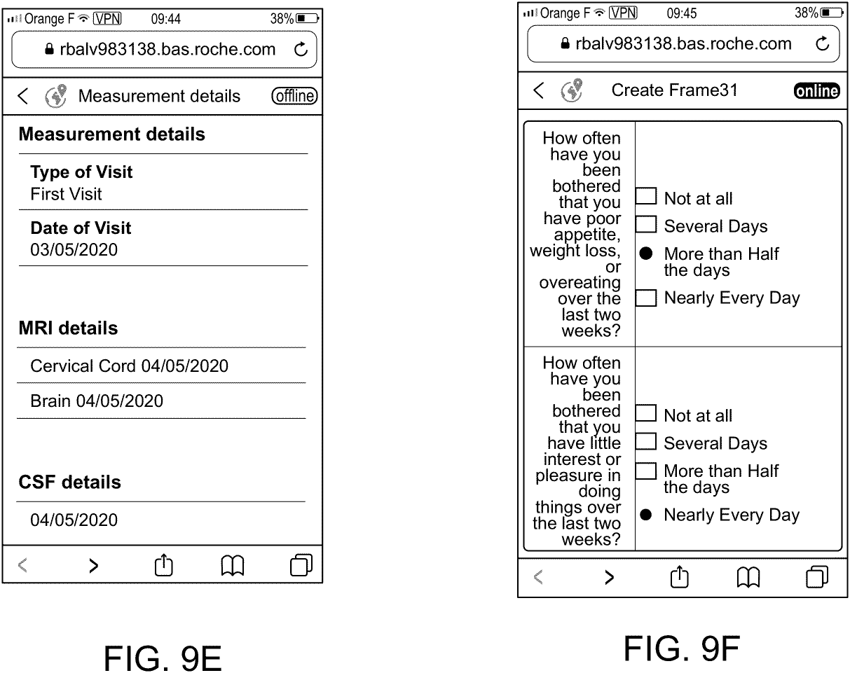

FIGS. 9A-9F depict exemplary interfaces configured to receive inputs to build a multiple-sclerosis record data store. FIG. 9A shows an interface that includes multiple editable sections identifying information corresponding to a particular subject having possible, probable or confirmed multiple sclerosis. A “person” section 905 includes multiple identifiers of the subject (an identifier number and name), a current residence location, birth location, contact information (phone numbers, email address), insurance information, and a current status (indicating alive or deceased). Person section 905 further identifies a date on which the subject signed an informed consent, which permitted one or more care providers to upload information about the subject to a computing system providing the interface and for the uploaded information to be used in particular manners. A copy of the informed consent that was signed itself can be uploaded through another page of the interface, such that the agreement can be examined. Person section 905 further identifies an age of the subject, which can be automatically updated in time.

A “demography, medical history” section 910 includes indications of whether the subject has any of a set of other diseases, whether the subject has a family history of multiple sclerosis or another autoimmune disease, smoking history, alcohol consumption quantity characterization, employment and marital status, educational level and ethnicity.

A “condition occurrences” section 915 can identify a sub-type of MS with which the subject was diagnosed, a date on which it was estimated that the subject’s multiple sclerosis presented itself, and a disability score. Symptom characterizations can further be identified via interaction with condition occurrences section 915.

A measurements section 920 can include results from a clinical, laboratory or imaging assessment.

A treatment section 925 can identify each MS treatment, symptomatic treatment and non-pharmacological treatment received by the subject and dates corresponding to each treatment. Treatment section 925 may further identify one or more adverse events experienced by the subject in response to a treatment.

Interacting with treatment section 925 can allow a user to select one or multiple MS treatments (e.g.. alemtizumab, azatioprina, copaxone, deaclizumab, dimethyl fumarate, methotrexate, mitoxantrone, natalizumab, interfron, ocrelizumab, fingolimod, rituximab, teriflunomide, other, or no treatment); indicate any adverse-event information (e.g., if pathogen: bacterial/fungal/viral/parasitic/unknown; onset/resolution date of adverse event, whether the adverse event required hospitalization; CTCAE toxicity grade (1-5 and/or outcome); and/or identify one or more symptom treatments (e.g., prednisone, cortisone, dexametasone, hydroortisone, methylprednisolone, prednisone). The interface may further be configured to receive, for each MS and/or symptom treatment, a dose, unit, period, route (sub-cutaneous, intramuscular, intravenous, oral, other), start/end date and/or reason for ending the treatment. The user may further be able to identify one or more non-pharmacological treatments being used by the subject (e.g., physiotherapy, yoga, vaccination, other).

FIG. 9B shows an exemplary interface configured to receive results from an MRI assessment. Data entered through this interface will then become accessible via measurement section 920 of the interface depicted in FIG. 9A. As shown, the interface of FIG. 9B is configured to accept input that identifies a date of the examination; which central-nervous-system region was imaged; whether T1 images were collected; an indication (via the “T1 type” field) as to whether the T1 images were normal, typical of MS, or abnormal and atypical of MS; a count of lesions detectable in the T1 images; whether gadolinium was administered; whether T1 images collected after the gadolinium administration were normal, typical of MS, or abnormal and atypical of MS; and a count of the number of contrast-enhancing lesions. The interface is further configured to accept input indicting whether T2 images were collected; an indication (via the “T2 type” field) as to whether the T2 images were normal, typical of MS, or abnormal and atypical of MS and a count of lesions detectable in the T2 images. The interface is further configured to accept input indicating a number of each of multiple types of lesions that are rather characteristic of MS (infratentorial, juxtacortical, periventricular and contiguous) and to accept a number of black holes that are detected.

Another interface (not shown) that can receive data corresponding to measurement section 920 may be configured to receive haematology data. The haematology data can include metrics corresponding to red cell count, haemoglobin count, platelet count, white cell count, lymphocyte count, T cell count, CD4 T cell count, CD8 T cell count, CD19 B cell count, NK cell count, neutrophil count, monocyte count, eosinophil count, and/or basophil count.

One or more other interfaces (not shown) that can receive data corresponding to measurement section 920 may be configured to receive blood chemistry data, thyroid-function data, serological data and/or auto-antibody test data. Blood chemistry data can characterize total protein count, albumin count, SGOT / AST count, SGPT / ALT count, gamma-GT count, bilrubin count, alkalin phosphate count, calcium count, urea count, uric acid count, creatine count, amylase count, lipase count, and/or Vitamin D levels. Thyroid function data can characterize levels of T3, T4, TSH, anti-microsomal antibodies, and/or anti-thryoglobulin antibodies. Serological data can indicate a presence of anti-JC virus, a presence of JC virus DNA in a subject’s urine, a presence of HBV antigen, a presence of anti-HCV, a presence of anti-HIV, a presence of anti-varicella, a result from a quantiferon gold test, a result from a mantoux test, a presence of neutralizing anti-IFN, a presence of neuralizing anti-natalizumab, and/or a result of a pregnancy test. Auto-antibody data can indicate presence or absence of NMO-IgG, anti-MOG, ANA, anti-mitcohndrial, anti-parietal cell, ASMA, anti-Ro, La, Sm, RNP, Sci-70, Jo1, anti-DNA, ANCA, anti-LKM, anticardiolipin, LAC, and/or anti-transglutaminase.

FIG. 9C shows an exemplary interface configured to receive results from an MRI assessment. Data entered through this interface will then become accessible via measurement section 920 of the interface depicted in FIG. 9A. As shown, the interface of 9C is configured to accept input that identifies a date on which cerebrospinal fluid was collected from the subject; whether the CSF was determined to correspond to a normal sample, a sample typical of MS, an abnormal sample also atypical of MS or a sample corresponding to trauma; whether oligoclonal bands were detected in the CSF, a quantity of oligoclonal bands detected, whether JC virus DNA was detected in the CSF; and an IgG index.

FIG. 9D shows an exemplary interface configured to receive results from evoked potentials. Evoked potentials can include sensory evoked potentials generated in response to somatosensory stimuli (SSEP), evoked potentials generated in response to auditory stimuli (BAEP), evoked potentials generated in response to visual stimuli (VEP) or motor evoked potentials (MEPs). The potentials may be detected at corresponding cortical areas on the right or left side of the body. The interface depicted in FIG. 9D is configured to identify whether each of multiple types of evoked potentials was normal, abnormal or unknown.

Each of the interfaces detected in FIGS. 9A-9D are accessible to a care provider. Other interfaces are further availed to the subject diagnosed with MS. FIG. 9E shows a subject-facing interface that includes select details about the types of data available in the subject’s record. FIG. 9F shows exemplary survey questions that are also accessible to the subject. Various questions may be presented at different times to the subject, and a notification can be presented that indicates that new questions are available. The questions may correspond to wellness-oriented questions that can be used to determine a wellness index for the subject.

While this Example provides illustrative interface configurations and data contemplated, other interface configurations and data variables are contemplated. For example, an interface may be configured to receive any type of disease characterization disclosed herein, any type of symptom characterization disclosed herein, any type of treatment characterization, any type of subject attribute disclosed herein, any type of clinical assessment disclosed herein, any type of medical-test or imaging result disclosed herein, and so on.

Thus, FIGS. 9A-9F exemplify interfaces through which users can provide information that characterizes a subject’s attributes, diagnosis, treatment and other information. This information may be usable to support big-data analyses to assess efficacy of a particular treatment generally or in a particular circumstance. This information may further or alternatively be usable to generate a query and/or define processing to be conducted that use other records in order to generate predictions for an individual subject associated with the record pertaining to treatment responsiveness.

### V. Additional Considerations

Some embodiments of the present disclosure include a system including one or more data processors. In some embodiments, the system includes a non-transitory computer readable storage medium containing instructions which, when executed on the one or more data processors, cause the one or more data processors to perform part or all of one or more methods and/or part or all of one or more processes disclosed herein. Some embodiments of the present disclosure include a computer-program product tangibly embodied in a non-transitory machine-readable storage medium, including instructions configured to cause one or more data processors to perform part or all of one or more methods and/or part or all of one or more processes disclosed herein.

The terms and expressions which have been employed are used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the present invention as claimed has been specifically disclosed by embodiments and optional features, modification and variation of the concepts herein disclosed may be resorted to by those skilled in the art, and that such modifications and variations are considered to be within the scope of this invention as defined by the appended claims.

The present description provides preferred exemplary embodiments only, and is not intended to limit the scope, applicability or configuration of the disclosure. Rather, the present description of the preferred exemplary embodiments will provide those skilled in the art with an enabling description for implementing various embodiments. It is understood that various changes may be made in the function and arrangement of elements without departing from the spirit and scope as set forth in the appended claims.

Specific details are given in the following description to provide a thorough understanding of the embodiments. However, it will be understood that the embodiments may be practiced without these specific details. For example, circuits, systems, networks, processes, and other components may be shown as components in block diagram form in order not to obscure the embodiments in unnecessary detail. In other instances, well-known circuits, processes, algorithms, structures, and techniques may be shown without unnecessary detail in order to avoid obscuring the embodiments.

### VI. Additional Examples

A first example includes a method including receiving, at a cloud-based application server, a query that identifies a treatment of multiple sclerosis and querying a data store using an identifier of the treatment, the data store having been populating based at least in part on input received from a distributed set of care-provider entities. The first example’s method further includes receiving, in response to the query, a set of subject identifiers, wherein each subject identifier in the set of subject identifiers indicates that a subject corresponding to the subject identifier received the treatment. The first example’s method further includes – for each subject identifier of the set of subject identifiers – determining, based on data in the data store, a time at which the subject corresponding to the subject identifier initiated the treatment; and extracting, from one or more records associated with the subject identifier: one or more metrics indicative of an outcome of the treatment; and one or more subject attributes The extraction of the one or more metrics was been based at least in part on the time at which the treatment was initiated, and each of the one or more subject attributes reflects a characteristic of a record-corresponding subject or a result of a medical test. The first example’s method still further includes generating a predicted responsiveness of another subject to the treatment based on the extracted metrics and the extracted subject attributes; and outputting a result corresponding to the predicted responsiveness.

A second example includes the method of the first example, wherein, for each subject identifier of the set of subject identifiers: the data store includes a set of subject-associated snapshots (each of the set of subject-associated snapshots corresponding to a particular time and the subject corresponding to the subject identifier); each snapshot in a subset of the set of subject-associated snapshots is associated with the treatment; and the one or more metrics are extracted from the subset of the set of subject-associated snapshots.

A third example includes the method of the second example, wherein, for at least some of the set of subject-associated snapshots, at least one of the one or more metrics was defined to be a metric value from a different time prior to the particular time upon determining that the data store did not include another metric value for the subject associated with a time subsequent to the different time and not exceeding the particular time corresponding to the snapshot.

A fourth example includes the method of any of the first through third examples, wherein the one or more metrics indicative of an outcome of the treatment include one or more absolute or relative statistics based on MRI results.

A fifth example includes the method of any of the first through fourth examples, wherein the one or more metrics indicative of an outcome of the treatment include one or more absolute or relative statistics based on relapse reporting.

A sixth example includes the method of any of the first through fifth examples, wherein the one or more metrics indicative of an outcome of the treatment include one or more absolute or relative statistics based on progression assessments.

A seventh example includes the method of any of the first through sixth examples, wherein the one or more metrics indicative of an outcome of the treatment include one or more absolute or relative statistics based on disability assessments.

An eighth example includes the method of any of the first through seventh examples, and further including training a machine-learning model using the extracted metrics and extracted subject attributes, wherein the predicted responsiveness is generated using the trained machine-learning model.

A ninth example includes the method of any of the first through eighth examples, and further including segregating the set of subject identifiers into two or more groups using the metrics; and assigning the other subject to a group of the two or more groups based on other subject metrics associated with the other subject, wherein the predicted responsiveness is generated based on the group assignment.

A tenth example includes the method of any of the first through ninth examples, wherein the each of the one or more subject attributes includes an attribute of the subject associated with a time at which the subject began the treatment.

An eleventh example includes the method of any of the first through tenth examples, wherein the received query further includes one or more particular attributes of the other subject, and wherein the query is performed based on the one or more particular attributes.

A twelfth example includes the method of any of the first through eleventh examples, wherein the one or more particular attributes includes an identification of another treatment previously received by the other subject.

A thirteenth example includes the method of any of the first through twelfth examples, further including predicting, based on the result, that the treatment will effectively treat multiple sclerosis for the other subject; and treating the other subject with the treatment.

1. A method comprising:
receiving, at a cloud-based application server, a query that identifies a treatment of multiple sclerosis;
querying a data store using an identifier of the treatment, the data store having been populating based at least in part on input received from a distributed set of care-provider entities;
receiving, in response to the query, a set of subject identifiers, wherein each subject identifier in the set of subject identifiers indicates that a subject corresponding to the subject identifier received the treatment;
for each subject identifier of the set of subject identifiers:
determining, based on data in the data store, a time at which the subject corresponding to the subject identifier initiated the treatment; and
extracting, from one or more records associated with the subject identifier:
one or more metrics indicative of an outcome of the treatment; and
one or more subject attributes, wherein the extraction of the one or more metrics was based at least in part on the time at which the treatment was initiated, and wherein each of the one or more subject attributes reflects a characteristic of a record-corresponding subject or a result of a medical test;
generating a predicted responsiveness of another subject to the treatment based on the extracted metrics and the extracted subject attributes; and
outputting a result corresponding to the predicted responsiveness.

2. The method of claim 1, wherein, for each subject identifier of the set of subject identifiers:
the data store includes a set of subject-associated snapshots, each of the set of subject-associated snapshots corresponding to a particular time and the subject corresponding to the subject identifier;
each snapshot in a subset of the set of subject-associated snapshots is associated with the treatment; and
the one or more metrics are extracted from the subset of the set of subject-associated snapshots.

3. The method of claim 2, wherein, for at least some of the set of subject-associated snapshots, at least one of the one or more metrics was defined to be a metric value from a different time prior to the particular time upon determining that the data store did not include another metric value for the subject associated with a time subsequent to the different time and not exceeding the particular time corresponding to the snapshot.

4. The method of claim 1, wherein the one or more metrics indicative of an outcome of the treatment include one or more absolute or relative statistics based on MRI results.

5. The method of claim 1, wherein the one or more metrics indicative of an outcome of the treatment include one or more absolute or relative statistics based on relapse reporting.

6. The method of claim 1, wherein the one or more metrics indicative of an outcome of the treatment include one or more absolute or relative statistics based on progression assessments.

7. The method of claim 1, wherein the one or more metrics indicative of an outcome of the treatment include one or more absolute or relative statistics based on disability assessments.

8. The method of claim 1, further comprising:
training a machine-learning model using the extracted metrics and extracted subject attributes, wherein the predicted responsiveness is generated using the trained machine-learning model.

9. The method of claim 1, further comprising:
segregating the set of subject identifiers into two or more groups using the metrics; and
assigning the other to a group of the two or more groups based on other subject metrics associated with the other subject, wherein the predicted responsiveness is generated based on the group assignment.

10. The method of claim 1, wherein the each of the one or more subject attributes includes an attribute of the subject associated with a time at which the subject began the treatment.

11. The method of claim 1, wherein the received query further includes one or more particular attributes of the other subject, and wherein the query is performed based on the one or more particular attributes.

12. The method of claim 11, wherein the one or more particular attributes includes an identification of another treatment previously received by the other subject.

13. The method of claim 1, further comprising:
predicting, based on the result, that the treatment will effectively treat multiple sclerosis for the other subject; and
treating the other subject with the treatment.

14. A system comprising:
one or more data processors; and
a non-transitory computer readable storage medium containing instructions which, when executed on the one or more data processors, cause the one or more data processors to perform operations including:
receiving, at a cloud-based application server, a query that identifies a treatment of multiple sclerosis;
querying a data store using an identifier of the treatment, the data store having been populating based at least in part on input received from a distributed set of care-provider entities;
receiving, in response to the query, a set of identifiers, wherein each identifier in the set of subject identifiers indicates that a subject corresponding to the subject identifier received the treatment;
for each subject identifier of the set of subject identifiers:
determining, based on data in the data store, a time at which the subject corresponding to the identifier initiated the treatment; and
extracting, from one or more records associated with the subject identifier:
one or more metrics indicative of an outcome of the treatment; and
one or more subject attributes, wherein the extraction of the one or more metrics was based at least in part on the time at which the treatment was initiated, and wherein each of the one or more subject attributes reflects a characteristic of a record-corresponding subject or a result of a medical test;
generating a predicted responsiveness of another subject to the treatment based on the extracted metrics and the extracted subject attributes; and
outputting a result corresponding to the predicted responsiveness.

15. The system of claim 14, wherein, for each subject identifier of the set of subject identifiers:
the data store includes a set of subject-associated snapshots, each of the set of subject-associated snapshots corresponding to a particular time and the subject corresponding to the subject identifier;
each snapshot in a subset of the set of subject-associated snapshots is associated with the treatment; and
the one or more metrics are extracted from the subset of the set of subject-associated snapshots.

16. The system of claim 15, wherein, for at least some of the set of subject-associated snapshots, at least one of the one or more metrics was defined to be a metric value from a different time prior to the particular time upon determining that the data store did not include another metric value for the subject associated with a time subsequent to the different time and not exceeding the particular time corresponding to the snapshot.

17. The system of claim 14, wherein the one or more metrics indicative of an outcome of the treatment include:
one or more absolute or relative statistics based on MRI results;
one or more absolute or relative statistics based on relapse reporting;
one or more absolute or relative statistics based on progression assessments; or
one or more absolute or relative statistics based on disability assessments.

18. A computer-program product tangibly embodied in a non-transitory machine-readable storage medium, including instructions configured to cause one or more data processors to perform operations comprising:
receiving, at a cloud-based application server, a query that identifies a treatment of multiple sclerosis;
querying a data store using an identifier of the treatment, the data store having been populating based at least in part on input received from a distributed set of care-provider entities;
receiving, in response to the query, a set of subject identifiers, wherein each subject identifier in the set of subject identifiers indicates that a subject corresponding to the subject identifier received the treatment;
for each subject identifier of the set of subject identifiers:
determining, based on data in the data store, a time at which the subject corresponding to the identifier initiated the treatment; and
extracting, from one or more records associated with the subject identifier:
one or more metrics indicative of an outcome of the treatment; and
one or more subject attributes, wherein the extraction of the one or more metrics was based at least in part on the time at which the treatment was initiated, and wherein each of the one or more subject attributes reflects a characteristic of a record-corresponding subject or a result of a medical test;
generating a predicted responsiveness of another subject to the treatment based on the extracted metrics and the extracted subject attributes; and
outputting a result corresponding to the predicted responsiveness.

19. The computer-program product of claim 18, wherein, for each subject identifier of the set of identifiers:
the data store includes a set of subject-associated snapshots, each of the set of subject-associated snapshots corresponding to a particular time and the subject corresponding to the subject identifier;
each snapshot in a subset of the set of subject-associated snapshots is associated with the treatment; and
the one or more metrics are extracted from the subset of the set of subject-associated snapshots.

20. The computer-program product of claim 19, wherein, for at least some of the set of subject-associated snapshots, at least one of the one or more metrics was defined to be a metric value from a different time prior to the particular time upon determining that the data store did not include another metric value for the subject associated with a time subsequent to the different time and not exceeding the particular time corresponding to the snapshot.
